(19) United States
(12) Patent Application Publication
(10) Pub. No.: US 2018/0116990 A1
(43) Pub. Date: May 3, 2018

(54) COMPOSITION OF BIOACTIVE LIPIDS AND METHODS OF USE THEREOF

(71) Applicant: UND Life Sciences, LLC, Battleground, WA (US)

(72) Inventor: Undurti Narasimha Das, Battleground, WA (US)

(21) Appl. No.: 15/724,858

(22) Filed: Oct. 4, 2017

Related U.S. Application Data
(60) Provisional application No. 62/496,040, filed on Oct. 4, 2016.

(51) Int. Cl. A61K 31/202; A61K 47/02; A61K 47/10; A61K 47/42; C07K 16/22; A61K 47/68; A61K 31/573; A61P 27/02; A61K 9/00

(52) U.S. Cl. CPC A61K 31/202; A61K 47/02; A61K 47/10; A61K 47/42; A61K 9/0048; A61K 47/6803; A61K 31/573; A61P 27/02; C07K 16/22

(57) ABSTRACT

The present application is generally directed to compositions comprising a bioactive lipid, a stabilizing agent, a solution, and ethanol. Methods associated with the preparation and use of such compositions, for example, for treating, preventing, or reversing diabetic retinopathy, age-related macular degeneration, retinopathy of prematurity in children, or diabetic macular edema in a subject in need thereof, are also provided.

### BACKGROUND

### Technical Field

Embodiments of the present invention generally relate to compositions comprising a bioactive lipid, a stabilizing agent and ethanol, as well as methods for use and preparation of the same.

### Description of the Related Art

The polyunsaturated fatty acids (PUFAs) are fatty acids having at least two carbon-to-carbon double bonds in a hydrophobic hydrocarbon chain which typically includes between 16 and 24 carbon atoms and terminates in a carboxylic acid group. The PUFAs are classified in accordance with a short hand nomenclature which designates the number of carbon atoms present (chain length), the number of double bonds in the chain and the position of double bonds nearest to the terminal methyl group. The notation “a:b” is used to denote the chain length and number of double bonds, respectively, and the notation “n-x” is used to describe the position of the double bond nearest to the methyl group. There are 4 independent families of PUFAs, depending on the parent fatty acid form from which they are synthesized. They are:

The “n-3” series derived from alpha-linolenic acid (ALA, 18:3, n-3).

The “n-6” serried derived from linoleic acid (LA, 18:2, n-6).

The “n-9” series derived from oleic acid (OA, 18:1, n-9).

The “n-7” series derived from palmitoleic acid (PA, 16:1, n-7).

The parent fatty acids of the n-3 and n-6 series cannot be synthesized by the mammals, and hence they are often referred to as “essential fatty acids” (EFAs). Because these compounds are necessary for normal health but cannot be synthesized by the human body, they must be obtained through the diet.

It is believed that both LA and ALA are metabolized by the same set of enzymes. LA is converted to gamma-linolenic acid (GLA, 18:3, n-6) by the action of the enzyme delta-6-desaturase (d-6-d), and GLA is elongated to form di-homo-GLA (DGLA, 20:3, n-6), the precursor of the 1 series of prostaglandins. The reaction catalyzed by d-6-d is the rate limiting step on the metabolism of EFAs. DGLA can also be converted to arachidonic acid (AA, 20:4, n-6) by the action of the enzyme delta-5-desaturase. AA forms the precursor of 2 series of prostaglandins (PGs), thromboxanes (TXs) and the 4 series leukotrienes (LTs). ALA is converted to eicosapentaenoic acid (EPA, 20:5, n-3) by d-6-d and d-5-d. EPA forms the precursor of the 3 series of PGs, TXs and 5 series of leukotrienes. EPA can be converted to docosahexaenoic acid (DHA, 22:6, n-3) and, in turn, DHA could be retroconverted to EPA. Conjugated linoleic acid (CLA; 18:2) is a group of isomers (mainly 9-cis, 11-trans and 10-trans, 12-cis) of linoleic acid. CLA is the product of rumen fermentation and can be found in the milk and muscle of ruminants {(see, e.g., Brodie et al. (1999), J. Nutr. 129: 602-6; Visonneau et al. (1997), Anticancer Res. 17: 969-73)} LA, GLA, DGLA, AA, ALA, EPA and DHA and CLA are all PUFAs, but only LA and ALA are EFAs. But, several actions of EFAs are also brought about by GLA, DGLA, AA, EPA and DHA and hence, are also called as “conditional EFAs” and hence, for all practical purposes the words EFAs and PUFAs are used interchangeably.

In addition, AA, EPA, and DHA also give rise to anti-inflammatory compounds such as lipoxins (from AA) and resolvins (from EPA and DHA) and resolvins, protectins and maresins (from DHA) that have potent anti-inflammatory actions (see FIGS. 1-5).

PUFA (AA, EPA and DHA and other fatty acids) are not only metabolized by COX and LOX enzymes but also by CYP-450 resulting in the formation of various metabolites including epoxyeicosatrienoic acids (EETs), hydroxyeicosatetraenoic acids (HETEs) and hydroperoxyeicosatetraenoic acids (HPETEs) that are known to actively participate in the inflammatory and other cellular processes. Most of these are intermediary metabolites involved in the prostanoid synthesis. COX-2 metabolizes free AA to form 11R, 15S-HPETE which is further reduced to 11R,15-HETE by peroxidase and is converted to 11- and 15-oxo-EET. EPA and DHA also undergo similar metabolic fates by cytochrome P450 as that of AA to form various products (see FIG. 6).

It is known in the art that certain PUFAs and/or their metabolites such as prostaglandins, leukotrienes, thromboxanes, lipoxins, resolvins, protectins and maresins regulate free radical generation, have anti-inflammatory activity, modulate immune response and thus, either enhance or inhibit endothelial cell migration, proliferation, and maturation proliferation, and regulate angiogenesis. In this context, it is noteworthy that lipoxins, resolvins, protectins and maresins inhibited leukotriene D4 and vascular endothelial growth factor (VEGF)-stimulated proliferation and angiogenesis both in vitro and in vivo by decreasing VEGF-stimulated VEGF receptor 2 (KDR/FLK-1) phosphorylation and downstream signaling events including activation of phospholipase C-γ, ERK1/2, and Akt.

Despite the known activity of lipoxins, resolvins, protectins and maresins as anti-inflammatory and modulators of immunity and anti-angiogenic actions, they have yet to be successfully used in methods for treatment of diabetic retinopathy (DR), retinopathy of prematurity in children, and age-related macular degeneration (AMD) at least in part because of the difficulty associated with formulating, difficulty in not knowing what concentrations these molecules to be used and in which ratio they could/should be mixed (for example in which ratio lipoxins, resolvins, protectins and maresins are to be mixed to elicit maximum beneficial action) and delivering them. Accordingly, there is a need in the art for improved formulations of lipoxins, resolvins, protectins and maresins and methods for their use in treatment of various diseases, including diabetic retinopathy (DR), retinopathy of prematurity in children, and age-related macular degeneration (AMD). The present invention provides these and other related advantages.

Despite the known activity of lipoxins, resolvins, protectins and maresins, they have yet to be successfully used in methods for treatment of DR, DME, AMD and retinopathy of prematurity, at least in part because of the difficulty associated with formulating and delivering them. Accordingly, there is a need in the art for improved formulations of lipoxins, resolvins, protectins and maresins and methods for their use in treatment of various diseases, including DR, DME, AMD and retinopathy of prematurity. The present invention provides these and other related advantages.

### BRIEF SUMMARY

In brief, embodiments of the present invention provide a pharmaceutical composition which is capable of selectively preventing and/or reversing DR, DME, AMD and retinopathy of prematurity when administered. In certain embodiments the composition comprises a LXA4, resolvin, protectin and/or maresin or a pharmaceutically acceptable salt or derivative thereof, a stabilizing agent, saline or phosphate buffered saline, and ethanol wherein the concentration of the stabilizing agent and ethanol. For example, in some embodiments the concentration of ethanol in the composition ranges from 0.0001% to 0.01% v/v. In other embodiments, the LXA4, resolvin, protectin and/or maresin is in the free acid form.

Accordingly, certain embodiments of the present invention provide a composition comprising:

one or more bioactive lipid, salt or derivative thereof, the bioactive lipid selected from the group consisting of a lipoxin, a resolvin, a protectin, and a maresin;

a stabilizing agent;

a solution selected from the group consisting of saline and phosphate buffered saline; and

ethanol;

wherein:

the concentration of the stabilizing agent and ethanol does not interfere with the anti-inflammatory, immunomodulatory and anti-angiogenic activity of the bioactive lipid.

In certain embodiments of the present invention provide a method for treating, preventing, or reversing diabetic retinopathy, age-related macular degeneration, retinopathy of prematurity in children, or diabetic macular edema in a mammal in need thereof, the method comprising administering to the mammal a therapeutically effective amount of a composition according to any of the embodiments described herein.

In other embodiments a method for preparing the pharmaceutical composition comprising, dissolving LXA4, resolvins, protectins, and/or maresins in ethanol to make a first mixture and diluting the first mixture in saline or phosphate buffered saline wherein the final concentration of ethanol ranges from 0.0001% to 0.01% is provided.

Accordingly, certain specific embodiments of the present invention provide a method for preparing a composition comprising:

dissolving one or more bioactive lipid, salt or derivative thereof, the bioactive lipid selected from the group consisting of a lipoxin, a resolvin, a protectin, and a maresin in ethanol to make a first mixture;

diluting the first mixture in a solution comprising saline or phosphate buffered saline thereby forming a second mixture, the second mixture having a concentration of ethanol ranging from 0.0001% to 0.01%; and

adding a stabilizing agent.

These and other aspects of the invention will be apparent on reference to the following detailed description.

### BRIEF DESCRIPTION OF THE DRAWINGS

FIG. 1 demonstrates the metabolism of essential fatty acids (EFAs: LA and ALA) and their conversion to their respective polyunsaturated fatty acids such as AA, EPA and DHA and formation of their various metabolites such prostaglandins, leukotrienes, thromboxanes, as lipoxins, resolvins, protectins, and maresins.

FIG. 2 shows the formation of prostaglandins (PGs), leukotrienes (LTs) and lipoxins (LXs) from arachidonic acid.

FIG. 3 shows the scheme of formation of resolvinE1 (RvE1) from EPA.

FIG. 4 shows the scheme of formation of resolvins from EPA and resolvins, protectins and maresins from DHA.

FIG. 5 shows various anti-inflammatory and immunomodulatory actions of LXA4, resolvins, protectins and maresins.

FIG. 6 shows the formation of various products from AA, EPA and DHA through cytochrome P450 enzymes.

FIG. 7A shows the normal retina (macula shown within the black circle)

FIG. 7B shows the histological section of normal retina, with photoreceptors (black arrows), retinal pigment epithelium (white arrow), and the choroid (red arrow).

FIG. 7C shows cross-sectional image of the retina generated by optical coherence tomography (OCT), an imaging technique that allows for real-time, non-invasive visualization of retinal architecture.

FIG. 8A shows medium-size drusen (arrow)

FIG. 8B shows a large drusen (arrow) in a patient with intermediate AMD.

FIG. 9 depicts a diagram showing the role of reactive oxygen species, VEGF, and PEDF in diabetic retinopathy;

FIG. 9A shows normal retina and FIG. 9B shows retina from a patient with proliferative diabetic retinopathy (PDR).

FIG. 9B shows some of the features of PDR in which there is increased production of VEGF, decreased production of PEDF, enhanced generation of reactive oxygen species, new vessel formation, enhanced expression of VEGFR-2 occurs and choriocapillaries are increased.

FIG. 10 shows effect of various bioactive lipids on angiogenesis induced by VEGF. It is evident from these results that all bioactive lipids are effective in suppressing VEGF-induced angiogenesis. Of all, LXA4 is the most effective and a combination of lipoxin, resolvin, protectin and maresin when used in a combination of 1:1:1:1, best results were seen. C=Control; T=positive test control taken as 100%; RSv=Resolvin E1; PRt=Protectin D1; MaR=Maresin; L+R+P+M=LXA4=resolvin D1+Protectin D1+Maresin in 1:1:1:1 ratio.

FIG. 11 shows the effect of various bioactive lipids against STZ-induced cytotoxic action on vascular endothelial cells in vitro. Of all, LXA4 is the most effective and a combination of lipoxin, resolvin, protectin and maresin when used in a combination of 1:1:1:1, best results were seen. Thus, bioactive lipids possess cytoprotective action. C=Control; STZ=Streptozotocin-induced cytotoxicity taken as positive control taken as 100%; RSv=Resolvin E1; PRt=Protectin D1; MaR=Maresin; L+R+P+M=LXA4=resolvin D1+Protectin D1+Maresin in 1:1:1:1 ratio.

FIG. 12 shows the effect of bioactive lipids on the production of IL-6 and TNF-α by human macrophage cells in vitro stimulated with LPS in vitro. Of all, LXA4 is the most effective and a combination of lipoxin, resolvin, protectin and maresin when used in a combination of 1:1:1:1, best results were seen. Thus, bioactive lipids possess potent antiinflammatory action. C=Control; LPS stimulated increase in IL-6 and TNF-α production taken as 100%; RSv=Resolvin E1; PRt=Protectin D1; MaR=Maresin; L+R+P+M=LXA4=resolvin D1+Protectin D1+Maresin in 1:1:1:1 ratio.

FIG. 13 shows the effect of bioactive lipids on the production of PGE2 and VEGF by adult human retinal pigment epithelial cells, ARPE 19 cells in vitro exposed to lipopolysaccharide (LPS). It is evident from these results that bioactive lipids suppress PGE2 and VEGF production and thus, have potent antiinflammatory and antiangiogenic actions. Of all, LXA4 is the most potent and a combination of lipoxin, resolvin, protectin and maresin when used in a combination of 1:1:1:1, best results were seen. C=Control; LPS=Positive control taken as 100%; RSv=Resolvin E1; PRt=Protectin D1; MaR=Maresin; L+R+P+M=LXA4=resolvin D1+Protectin D1+Maresin in 1:1:1:1 ratio.

FIG. 14 shows the effect of bioactive lipids on leukocyte migration and adherence stimulated by LPS in vitro. It is evident from these results that bioactive lipids suppress leukocyte migration and adherence stimulated by LPS. Of all, LXA4 is the most potent and a combination of lipoxin, resolvin, protectin and maresin when used in a combination of 1:1:1:1 gave the best results. C=Control; LPS=positive control taken as 100%; RSv=Resolvin E1; PRt=Protectin D1; MaR=Maresin; L+R+P+M=LXA4=resolvin D1+Protectin D1+Maresin in 1:1:1:1 ratio.

FIG. 15 shows the effect of bioactive lipids on the production of BDNF by adult human retinal pigment epithelial cells, ARPE 19 cells in vitro exposed to lipopolysaccharide (LPS). Of all, resolvins, protectins and maresins are more potent than LXA4 in enhancing BDNF production, a known cytoprotective molecule and a combination of lipoxin, resolvin, protectin and maresin when used in a combination of 1:1:1:1 gave the best results. C=Saline control taken as 100%; LPS=LPS-induced decrease in BDNF production; RSv=Resolvin E1; PRt=Protectin D1; MaR=Maresin; L+R+P+M=LXA4=resolvin D1+Protectin D1+Maresin in 1:1:1:1 ratio.

FIG. 16 shows the effect of bioactive lipids on DR in experimental animals. The degree of DR in control is taken as 100% which received only the vehicle. All bioactive lipids are able to suppress/reverse/arrest DR to a significant degree. But in combination with anti-VEGF antibody, bioactive lipids are more effective. Similar results were obtained with AMD, DME and retinopathy of prematurity. AVA=Anti-VEGF antibody; LRPM=Lipoxin A4+resolvin+protectin+maresin; AVA+LRPM=Anti-VEGF antibody+Lipoxin A4+resolvin+protectin+maresin.

FIG. 17 shows the effect of different concentrations of albumin on the stability of LXA4 in vitro. Y axis shows the percentage of LXA4 in the solution tested. C=Control (100%); Concentration of LXA4 used in the test system is 10 μg to which different concentrations of albumin is added and after a specific period of incubation the concentration of LXA4 present in the solution is tested. It is seen that when the % of albumin exceeded 0.01%, the stability of LXA4 is decreased. But, when the concentration of albumin is between 0.01% to 0.001% LXA4 is stable and active and is released in optimum amounts to bring about its actions. The test system used: LXA4 is dissolved in 0.01% ethanol (pH 7.4) at 37° C. to which different concentrations of albumin is added and at the end of incubation time the concentration of LXA4 present is tested to know whether albumin would influence the stability of LXA4 in the solution.

FIG. 18 shows the effect of albumin on the stability of LXA4 at different time(s) in vitro. Concentration of LXA4 tested 100 ng. Y axis shows the percentage of LXA4 in the solution tested. C=Control (100%) (expected). Various doses of albumin were added to LXA4 solution in saline/PBS/ethanol and the stability of LXA4 was tested at the end of different periods of incubation in days. Results were best when ethanol concentration in the solution is between 0.01 to 0.0001% and albumin concentration is between 0.01 to 0.0001%.

### DETAILED DESCRIPTION

Embodiments of this invention relate to compositions and the efficacious use of lipoxin A4 (LXA4), resolvins, protectins and maresins. Embodiments of these compositions are obtained using a distinct method of solubilizing and delivery is accomplished intra-vitreally directly to the eye resulting in no side-effect and without loss of potency. Embodiments of the compositions selectively suppress pathological angiogenesis that occurs in diseases such as retinopathy of prematurity in children, diabetic retinopathy, and age-related macular degeneration in which cell proliferation and angiogenesis plays a dominant role. In some embodiments, intravitreal administration of LXA4, resolvins, protectins and maresins selectively and effectively suppressed and ameliorated pathological retinopathy prematurity in children, diabetic retinopathy, and age-related macular degeneration more effectively than the current anti-VEGF and corticosteroid therapies.

Growth factors are proteins secreted by several types of cells in the body that have potent actions on cell proliferation, migration, and differentiation. These growth factors bind to their respective receptors that in turn lead to the activation of transmembrane receptor tyrosine kinases. Examples of these growth factors include: epidermal growth factor (EGF), VEGF, fibroblast growth factor (FGF), platelet derived growth factor (PDGF), hepatocytes growth factor (HGF), placental growth factor (PIGF), and tyrosine kinase receptor erbB2, also known in humans as Her 2. These growth factors also stimulate angiogenesis. Of all the growth factors, VEGF is especially important since it facilitates angiogenesis and neovascularization that is relevant to development of diabetic retinopathy (DR), retinopathy of prematurity in children, and age-related macular degeneration (AMD).

The microcirculatory problems people with diabetes mellitus can cause retinal ischemia, which results in release of excess of VEGF that causes neoangiogenesis that ultimately leads to DR. As such, emphasis has been put on the role of VEGF in pathological angiogenesis and anti-VEGF therapies have been developed and emphasized. Some of these anti-VEGF therapies include: bevacizumab (Avastin®), ranibizumab (Lucentis®), sunitinib (Sutent®), sorafenib (Nexavar®), axitinib, pazopanib and pegaptanib (Macugen®).

However, treatment of these conditions is inadequate. Clinical trials indicate that still a substantial number of patients (almost 66%) remain without any benefit and are in need of better drugs for their condition. Furthermore, there are substantial side effects noted with the use of the currently available anti-angiogenic drugs such as pegaptanib sodium (Macugen®) for partial blockage of VEGF-A, or ranibizumab (Lucentis®) and bevacizumab (Avastin®).

The exact pathogenesis of DR is still debated. But some of the mechanisms that are involved in the onset and progression of DR include: (i) increased activity of aldose reductase that leads to enhanced production of sorbitol that may cause osmotic or other cellular damage. But several aldose reductase inhibitors failed to produce any significant benefits; (ii) there is evidence of low-grade inflammation in DR as evidenced by increased adherence of leukocytes to capillary endothelium that can result in decrease in blood flow and increase in hypoxia that results in breakdown of blood-retinal barrier and enhanced macular edema. In view of this aspirin was tried but of no benefit. It was noted that anti-VEGF antibody is of some benefit and so also benefit of intravitreal triamcinolone was reported. In fact, some studies suggested that intravitreal injection of triamcinolone showed better results in reducing DME and improvement of visual acuity than that of bevacizumab, an anti-VEGF antibody, suggesting that the pathogenesis of DME is not attributable to VEGF-dependency, but is also attributable to other mechanisms that are suppressed by corticosteroids (Shimura M, Nakazawa T, Yasuda K, Shiono T, Iida T, Sakamoto T, Nishida K. Comparative therapy evaluation of intravitreal bevacizumab and triamcinolone acetonide on persistent diffuse diabetic macular edema. Am J Ophthalmol 2008; 145: 854-861).

A combination of bevacizumab and triamcinolone does not have any additive benefit implying that both bevacizumab and triamcinolone are working through the same common pathway and no additional benefit is evident and/or pathways other than VEGF and inflammatory events are involved in DR/DME.

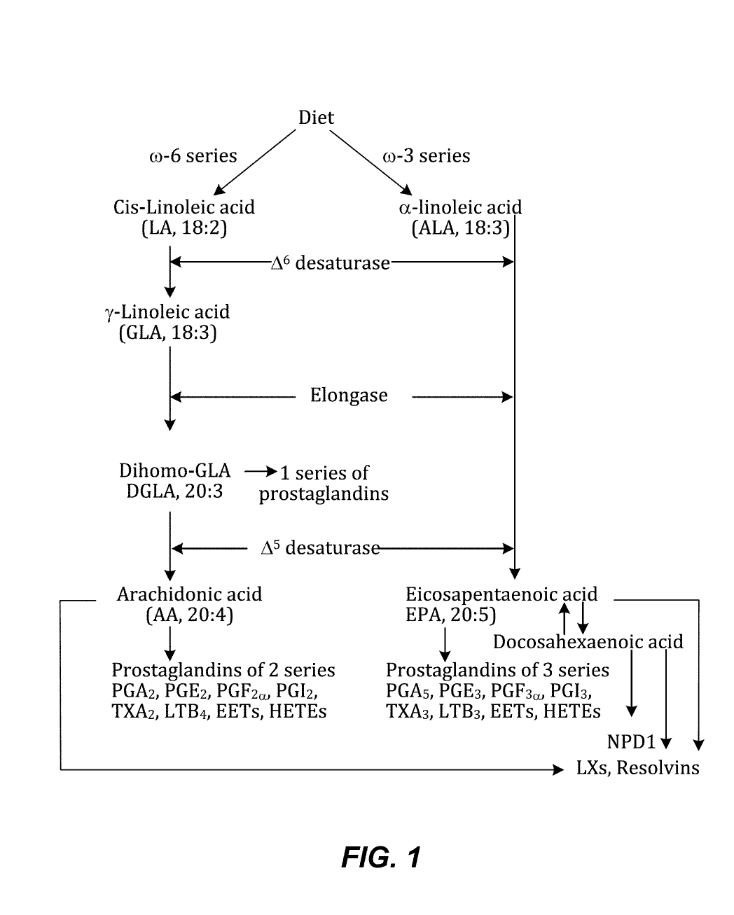

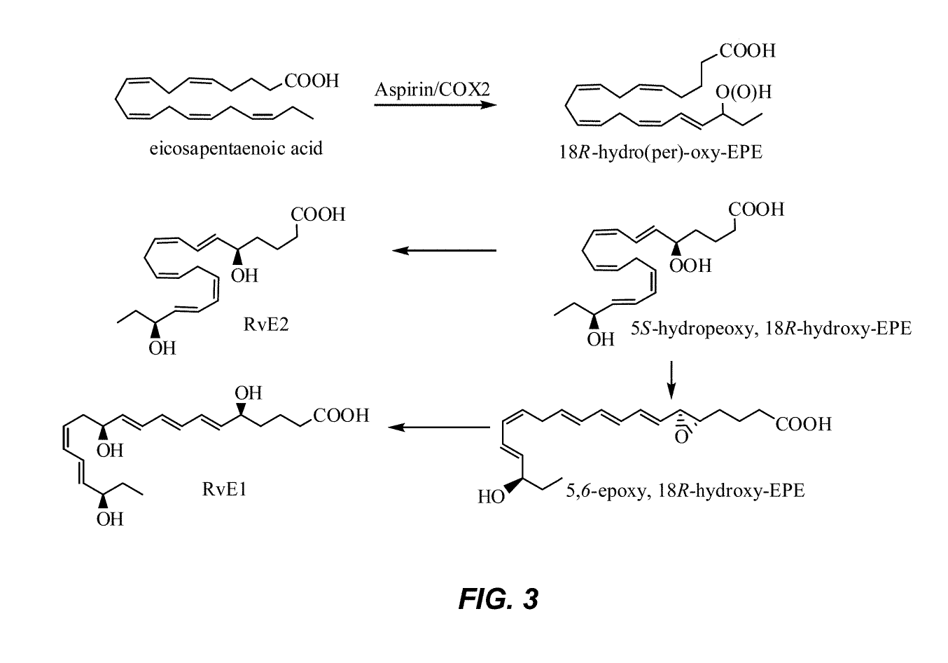

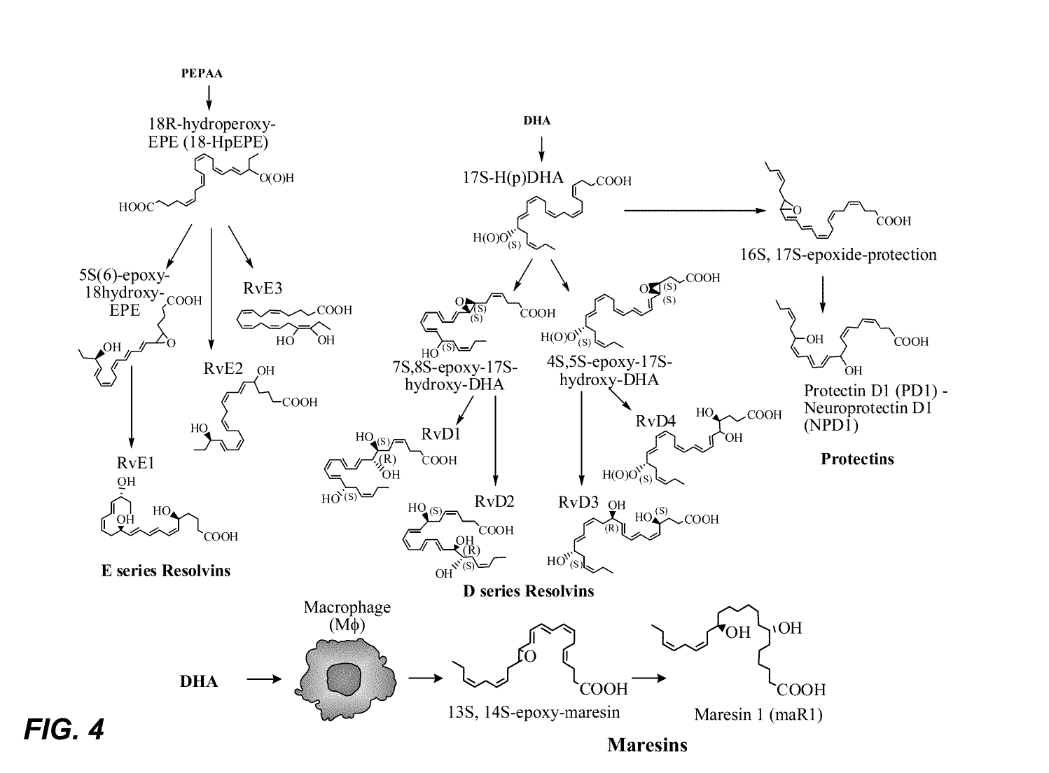

In this context, it is interesting to note that corticosteroids block Δ⁶ and Δ⁵ desaturases (delta-6- and adleta-5 desaturases), enzymes that are essential for the conversion of dietary LA and ALA to their respective long-chain fatty acid metabolites namely AA (from LA) and EPA and DHA (from ALA), that are precursors of LXA4 (from AA), resolvins (from EPA and DHA) and protectins and maresins (from DHA) (see FIGS. 1-4). Furthermore, corticosteroids block COX-2 (cyclo-oxygenase-2) and lipoxygenase (LOX) enzymes that are needed for the synthesis of prostaglandins, thromboxanes and leukotrienes, which are pro-inflammatory eicosanoids. Thus, triamcinolone and other corticosteroids work as potent anti-inflammatory compounds in the beginning by blocking the formation of PGs, LTs and TXs. But, due to their inhibitory actions on desaturases enzymes, the cell concentrations of AA, EPA and DHA would decrease. As a result, cell content of AA, EPA and DHA will be low and so the formation of LXA4, resolvins, protectins, and maresins will be reduced due to their precursor (AA, EPA and DHA) deficiency. Thus, the anti-inflammatory action of corticosteroids is short lived and prolonged use of steroids would lead to decreased formation of LXA4, resolvins, protectins and maresins that are needed for long-term anti-inflammatory action and resolution of inflammation. Thus, the initial dramatic anti-inflammatory action of corticosteroids fails to produce long-term benefit due to failure of formation of adequate amounts of potent endogenous anti-inflammatory and proresolution compounds: LXA4, resolvins, protectins and maresins.

In another study, Soheilian et al (Retina 2012; 32: 314-321) showed that in terms of vision improvement, the significant superiority of the IVB over the combined IVB/IVT and macular laser photocoagulation (MPC) treatment that had been observed at month 6 did not sustain up to 24 months. This means that although IVB treatment may be a better choice than two other options in short term, the magnitude of this beneficial effect diminishes over time. This result once again confirms the short-term beneficial action of anti-VEGF and triamcinolone treatment for DME and DR but fails to show sustained long-term benefit that is desired. This once again calls for a better understanding of the pathobiology of DME and DR (and also that of AMD and retinopathy of prematurity) and one need to go beyond VEGF and corticosteroids and develop newer therapeutic approaches in their prevention and management.

This is further supported by the recent report (Biomed Res Int 2015; 2015: 352487) that intravitreal triamcinolone monotherapy resulted in some improvement in vision. Treatment with threshold or subthreshold grid laser also resulted in minimal vision gain. Anti-VEGF therapy resulted in more significant visual improvement. In contrast and surprisingly treatment with pars plana vitrectomy and internal limiting membrane (ILM) peeling alone resulted in an improvement in vision greater than that observed with anti-VEGF injection alone. Thus, in this DME study, treatment with vitrectomy and ILM peeling alone resulted in the better visual improvement compared to other anti-VEGF and intravitreal triamcinolone therapies. (iii) Hyperglycemia enhances the activity of protein kinase C (PKC) that, in turn, upregulates VEGF and also active in downstream actions of VEGF following binding of VEGF to its cellular receptor (VEGFR). But clinical trials of a PKCβ isoform inhibitor in DR have been unsuccessful. For instance, in the studies combined, sustained moderate visual loss occurred in 10.2% of placebo-treated patients versus 6.1% of ruboxistaurin (RBX), a protein kinase C β inhibitor-treated patients (P=0.011). A≥15-letter gain occurred in 2.4% of placebo versus 4.7% of RBX eyes (P=0.021) and a≥15-letter loss occurred in 11.4% versus 7.4%, respectively (P=0.012). Diabetic macular edema was the probable primary cause of vision loss. Among eyes without focal/grid photocoagulation at baseline, fewer RBX group eyes (26.7%) required initial focal/grid photocoagulation versus placebo (35.6%; P=0.008) (Retina 2011; 31: 2084-2094). In another clinical trial, RBX was well tolerated and reduced the risk of visual loss but did not prevent DR progression (PKC-DRS Study Group, Diabetes 2005; 54: 2188-2197) and so no further efforts are being made to develop PKC inhibitors for DR and DME. (iv) Oxidative damage to enzymes and to other key cellular components is proposed to play a major role in DR and DME. Hyperglycemia enhances free radical generation that could initiate and perpetuate inflammation and apoptosis of pericytes of the retinal capillaries that occurs in DR. In view of this several anti-oxidants have been tried in DR but were found to be of limited value. (v) Long-standing hyperglycemia causes non-enzymatic glycation of various cellular proteins and leads to the formation of excess of advanced glycation end products (AGE) that may play a role in DR and DME. But clinical trials with aminoguanidine, an inhibitor of formation of AGE failed to show any favorable results in patients with DR. (vi) Hyperglycemia causes upregulation of inducible nitric oxide synthase (iNOS) leading to formation and release of excess of nitric oxide (NO) that may lead, in turn, to enhanced free radical generation leading to upregulation of VEGF. Aminoguanidine is known to suppress iNOS. But clinical trials with aminoguanidine have been disappointing. (vii) Increase in the apoptotic death of retinal capillary pericytes and endothelial cells is documented in DR and are considered as the initiating events in the development of DR. These events lead to reduction in blood flow to retina resulting in hypoxia that leads to increased production of VEGF that, in turn, is involved in DR. But, at present there are no specific drugs available to protect retinal capillary pericytes and endothelial cells. Anti-VEGF drugs are not specific enough to protect specifically retinal capillary pericytes and endothelial cells that may explain as to why anti-VEGF therapy is not very effective. (viii) Increased production of VEGF: Human retinal pigment epithelial cells and choroid have the ability to secrete VEGF whereas VEGF receptors are known to be present on the inner choriocapillaries. Increased retinal hypoxia and other mechanisms trigger the production of VEGF that induces breakdown of the blood-retinal barrier leading to macular edema, proliferation of retinal capillary cells and neovascularization. These evidences led to the use of anti-VEGF therapies for DMR and DR. But, unfortunately increased VEGF is not the only mechanism involved which may explain as to why anti-VEGF therapies are not very effective in DME and DR. (ix) Pigment epithelium derived factor (PEDF): is a pluripotent glycoprotein belonging to the serpin family and can stimulate several physiological processes such as angiogenesis, cell proliferation, and survival. PEDF plays a protective role in DR and there is accumulating evidence of the neuroprotective effect of PEDF (Elahy M, et al., J Endocrinol 2014; 222: R129-R139).

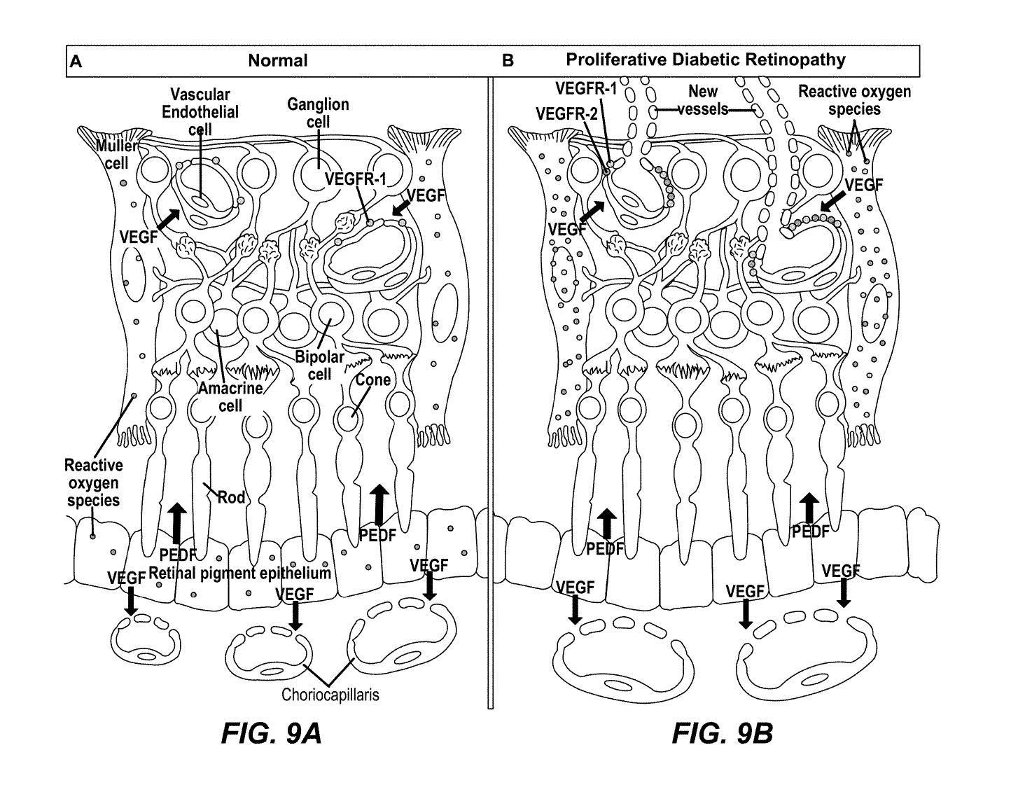

In a recent study, we found that the serum BDNF and LXA4 levels were significantly reduced in both non-proliferative diabetic retinopathy (NPDR) and proliferative diabetic retinopathy (PDR) cases compared to control. Serum IL-6 was significantly increased in the PDR group. BDNF showed a significant negative correlation with VEGF levels (r=−0.522, p<0.01) and positive correlation with IL-10 (r=0.67, p<0.05) in serum. A significant odds ratio for the serum BDNF (OR: 3.20, p=0.025) as well as serum IL-6 (OR: 1.244, p=0.042) indicated them as potential risk factors for progression of type 2 DM to DR. A significant decrease in both the LXA4 (p=0.013) and BDNF (p=0.0008) with increase in cytokines IL-6 and IL-10 levels were observed in the vitreous of PDR cases ((p=0.04, 0.01). In vitro studies showed that both LXA4 (10 nmol/L) and BDNF (500 pg) decreased the IL-6 levels (p=0.036, 0.0002), in LPS induced pro-inflammatory condition in ARPE 19 cells, thereby indicating their (LXA4 and BDNF) anti-inflammatory effect. No significant changes in the serum and vitreal PEDF levels were noted in DR patients (Kaviarasan K., et al., Metabolism 2015; 64: 958-966). This study reports that low serum BDNF and higher IL-6 levels are potential risk factors for DR in type 2 DM. This study supports the role of BDNF in modulating the pro- and anti-inflammatory cytokines, and low level of BDNF is associated with development of diabetic retinopathy. In this context, it is interesting to note that we also observed that LXA4 enhances the production of BDNF whereas BDNF enhances the synthesis and release of LXA4. PEDF was originally isolated from fetal retinal pigment epithelial cells but is now known to be synthesized elsewhere and throughout the body. PEDF promotes the differentiation of primitive cultured retinoblastoma cells into neuron-like structures. PEDF inhibits neovascularization. PEDF may be needed for maintaining the neural architecture of the retina. The role of various factors involved in the pathogenesis of DR is represented in FIG. 9. Despite these advances in the understanding of DR and DME, there is no effective therapy for them and anti-VEGF drugs could pass into the systemic circulation, which could potentially result in hypertension, proteinuria, increased cardiovascular events and impaired wound healing.

In addition, several polypeptide growth factors and their cell membrane receptors participate in the pathogenesis of DR. LXA4 is predominantly produced by retinal vascular endothelial cells. Retinal pigment epithelial cells predominantly produce resolvins and ganglion cells produce predominantly protectins and maresins. In addition, retinal vascular endothelial cells, retinal pigment epithelial cells and ganglion cells are all capable of producing LXA4, resolvins, protectins and maresins even though each type of cell predominantly produces one type of bioactive lipid as mentioned above.

LXA4, resolvins, protectins and maresins may augment each other's action and production such that a sequential and orderly production of these bioactive lipids occurs. For instance, in the initial stages of resolution of inflammation increased formation of LXA4 occurs, subsequently resolvins are produced to augment the resolution of inflammation and healing process to occur, this will be followed by enhanced production of protectins to protect ganglion cells, pigment epithelial cells and retinal capillary pericytes and endothelial cells. Maresins are produced at a later stage to help in tissue regeneration. In this sequence of production of these bioactive lipids, there will be overlap in the production of LXA4, resolvins, protectins and maresins. It also need to be understood that all bioactive lipids (LXA4, resolvins, protectins and maresins) possess anti-inflammatory, immunomodulatory and cytoprotective actions and their actions overlap each other's action.

In certain embodiments, the compositions showed anti-inflammatory and immunomodulatory action, suppressed endothelial cell migration, proliferation, and maturation needed for pathological angiogenesis; inhibited inflammatory responses including interleukin-6 (IL-6), tumor necrosis factor-a (TNF-α), interferon-y (IFN-γ) and IL-8 secretion as well as endothelial ICAM-1 expression and secretion and upregulated IL-10 production; inhibited leukotriene D4 (LTD4) and vascular endothelial growth factor (VEGF)-stimulated endothelial cell proliferation and angiogenesis and decreased the production of VEGF; protected ganglion cells, pigment epithelial cells and retinal capillary pericytes and endothelial cells and restored normal architecture of retina and surrounding tissues by inducing tissue regeneration and thus,

In some embodiments, a method for treatment of DR, AMD, DME and retinopathy of prematurity comprising identifying a neoangiogenic region and administering a therapeutically effective amount of a pharmaceutical composition consisting of lipoxins, resolvins, protectins and maresins in defined proportions and concentrations to a subject in need thereof wherein the composition selectively causes decrease or amelioration of inflammation, resolution of inflammation, prevention of progression of inflammation, prevents or reverses angiogenesis, protects ganglion cells, pigment epithelial cells, retinal capillary pericytes and endothelial cells and restores normal architecture of retina and surrounding tissues by inducing tissue regeneration and thus, prevents and ameliorates retinopathy of prematurity in children, diabetic retinopathy, and age-related macular degeneration.

Several polypeptide growth factors and their cell membrane receptors participate in the pathogenesis of DR. Of all, VEGF and its receptors (VEGFR-1 and VEGFR-2) and PEDF are considered to have a major role in DR. No receptors for PEDF have yet been identified. Both VEGF and PEDF are produced in the retinal pigment epithelial cells. Retinal neovascularization in DR and DME and AMD are always away from the retinal pigment epithelium and towards the vitreous space. Both VEGF and PEDF are also produced in retinal neurons and in glial cells. In the normal retina VEGFR-1 is the predominant VEGF receptor on the surface of retinal vascular endothelial cells, but in diabetes, VEGFR-2 appears on the endothelial cell plasma membrane. LXA4 is predominantly produced by retinal vascular endothelial cells. Retinal pigment epithelial cells predominantly produce resolvins and ganglion cells produce predominantly protectins and maresins. In addition, retinal vascular endothelial cells, retinal pigment epithelial cells and ganglion cells are all capable of producing LXA4, resolvins, protectins and maresins even though each type of cell predominantly produces one type of bioactive lipid as mentioned above. LXA4, resolvins, protectins and maresins may augment each other's action and production such that a sequential and orderly production of these bioactive lipids occurs. For instance, in the initial stages of resolution of inflammation increased formation of LXA4 occurs, subsequently resolvins are produced to augment the resolution of inflammation and healing process to occur, this will be followed by enhanced production of protectins to protect ganglion cells, pigment epithelial cells and retinal capillary pericytes and endothelial cells. Maresins are produced at a later stage to help in tissue regeneration. In this sequence of production of these bioactive lipids, there will be overlap in the production of LXA4, resolvins, protectins and maresins. It also need to be understood that all bioactive lipids (LXA4, resolvins, protectins and maresins) possess anti-inflammatory, immunomodulatory and cytoprotective actions and their actions overlap each other's action. These bioactive lipids may also act on stem cells and influence their survival, proliferation and differentiation as the situation demands.

In the following description, certain specific details are set forth in order to provide a thorough understanding of various embodiments of the invention. However, one skilled in the art will understand that embodiments of the invention may be practiced without these details.

Unless the context requires otherwise, throughout the present specification and claims, the word “comprise” and variations thereof, such as, “comprises” and “comprising” are to be construed in an open, inclusive sense that is as “including, but not limited to”.

Reference throughout this specification to “one embodiment” or “an embodiment” means that a particular feature, structure or characteristic described in connection with the embodiment is included in at least one embodiment of the present invention. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

Unless defined otherwise, all technical and scientific terms used herein have the same meaning as is commonly understood by one of skill in the art to which this invention belongs. As used in the specification and claims, the singular form “a”, “an” and “the” include plural references unless the context clearly dictates otherwise.

Retinal vascular anatomy, which is highly organized and easily visualized, and there is a close relationship between retinal vascular and neural structures in the form of shared radial orientation of blood vessels and ganglion cell axons, and in planar capillary plexuses that align precisely with horizontal neural and astrocytic laminae. In addition, vascularized and avascular compartments are strictly segregated in the retina; this feature is strikingly depicted in the human central retina, or fovea, which is entirely devoid of vessels. By contrast, pathological retinal angiogenesis as seen in DR and PDR is characterized by chaotically orientated and physiologically deficient vessels that do not conform to neuronal histology, which leads to vision-threatening exudation and hemorrhage. Thus, pathological retinal angiogenesis in the context of DR and proliferative retinopathy refers to chaotically orientated and physiologically deficient vessels, which lead to vision-threatening exudation and hemorrhages. By definition, it is proposed that DR refers to any abnormality in the retinal vasculature that deviates from normal.

“Polyunsaturated fatty acid” or “PUFA” refers to any acid derived from fats by hydrolysis, or any long-chain (at least 12 carbons) organic acid, having at least two carbon-to-carbon double bonds. Examples of PUFAs include but are not limited to linoleic acid, linolenic acid and arachidonic acid. Even though some embodiments of the invention specifically deals with PUFAs, it may be mentioned here that butyric acid, a short-chain fatty acid, was also found to have biological actions/functions on neuronal cells both in vitro and in vivo {Williams et al (2003) Proc Nutrition Soc 62: 107-115}. Hence, in the present definition of EFAs or PUFAs, the name of the short-chain fatty acid butyric acid (BA, 4:0) is included. Thus, the definition of EFAs/PUFAs is extended to those lipids that have various biological actions. Specifically, butyric acid is included. The other short-chain fatty acids such as formic acid, acetic acid, propionic acid, isobutyric acid, valeric acid and isovaleric acid are not included in this definition of “lipids that have biological actions.” Thus, as used herein, the definition of EFAs/PUFAs is extended to include not only LA, GLA, DGLA, AA, ALA, EPA and DHA but also BA. Lipoxins, resolvins, protectins and maresins that are derived from AA, EPA and DHA are labelled as “bioactive lipids” for the sake of simplicity and to refer to them together and are also included under the category of PUFA/PUFAs/EFAs for ease of expression.

“PUFA salt” refers to an ionic association, in solid or in solution, of a anionic form of a PUFA with a cation of a small organic group (e.g., ammonium) or a small inorganic group (e.g., an alkali metal). Salts include those between a PUFA and an alkali metal (e.g., lithium, sodium, potassium), and alkali earth metal (e.g., magnesium, calcium) or a multivalent transition metal (e.g., manganese, iron, copper, aluminum, zinc, chromium, cobalt, nickel).

“Intravitreal ” refers to any method of invasively or noninvasively injecting any drug or substance into the vitreal cavity of the eye. Invasive methods include direct injection using a needle, catheter, or any other device that can be inserted into the vitreal cavity that has a catheter/needle whose tip could be inserted into the vitreal cavity and/or releases bioactive lipids into the vitreal cavity. This definition of intravitreal also includes those measures that include inserting or instillation of nano particles or liposomes containing bioactive lipids to conjunctiva or cornea or delivered in the form of eye drops such that the bioactive lipids are ultimately delivered to vitreal cavity either directly or indirectly by diffusion from conjunctiva/cornea into the vitreal cavity. Noninvasive intravitreal injection method could include injecting into the conjunctiva or corneal or other parts of the eye that is “proximal ” with respect to vitreal cavity or region. Noninvasive methods of intravitreal injection may also include injecting into an area of eye as a result of which the said drug could reach the vitreal cavity or region. In both instances of methods of injection/instillation of eye drops in the form of nanoparticles or liposomes, if the composition is able to reach vitreal area or region or cavity in sufficient amounts is considered as noninvasive method of delivery of the drug to the vitreal cavity. Thus, for the purposes of definition, it is suggested that direct intravitreal injection of a composition into the vitreal cavity is considered as invasive method of delivery and when a composition is able to reach vitreal cavity or region in sufficient amounts even when it is injected/instilled into a proximal area is considered as an example of noninvasive method of delivery. This method of delivery of the drug includes placing a liposomal and/or nanoparticles of the drug applied to conjunctiva or injected into the vitreal cavity. Liposomal or nanoparticles of the drug may be delivered by direct injection into the vitreal cavity and/or placing a biodegradable membrane in which the drug has been incorporated for long term delivery of the said drug to the vitreal cavity. Such a liposomal and/or nanoparticles of the said drug incorporated in a biodegradable membrane or similar delivery system may also be injected or placed in the vitreal cavity for intermediate or long term delivery of the drug.

“Neoangiogenesis” or “neovascularization” refers to formation of new blood vessels. The terms “angiogenesis” and “neoangiogenesis” are used interchangeable to imply that new blood vessels are being formed to supply nutrients to a tissue. Angiogenesis is the physiological process through which new blood vessels form from pre-existing vessels. This is distinct from vasculogenesis, which is the de novo formation of endothelial cells from mesoderm cell precursors. The first vessels in the developing embryo form through vasculogenesis, after which angiogenesis is responsible for most, if not all, blood vessel growth during development and in disease. Angiogenesis is a normal and vital process in growth and development, as well as in wound healing and in the formation of granulation tissue. However, it is also a fundamental step in the transition of normal retinal angiogenesis to abnormal angiogenesis or neoangiogenesis or pathological angiogenesis seen in DR, AMD and hypoxic retinopathy of newborn (also called as retinopathy of prematurity).

“Lipoxin” refers to lipoxygenase interaction products, which are generally bioactive autacoid metabolites of arachidonic acid (AA). Lipoxins can be categorized as non-classic eicosanoids and members of the specialized pro-resolving mediator family of PUFA metabolites. Lipoxins include, for example, lipoxin A4 (LXA4), lipoxin B4 (LXB4) as well as epimers of the same (i.e., 15-epi-LXA4 and 15-epi-LXB4, respectively).

“Resolvin” refers to an autacoid that is a dihydroxy or trihydroxy metabolite of omega-3 fatty acids, including eicosapentaenoic acid (EPA), docosahexaenoic acid (DHA), docosapenaenoic acid (DPA) and clupanodonic acid. Resolvins are members of the specialized pro-resolving mediator class of PUFA metabolites. Resolvins include, for example, resolvin D1 and resolvin E1.

“Protectin” refers to an autacoid derived from unsaturated fatty acids which are generally characterized by the presence of a conjugated system of double bonds. Protectins are members of the class of specialized pro-resolving mediator class of PUFA metabolites and include, for example, neuroprotectins, protectin D1, 22-hydroxy NPD1, protectin DX, aspirin-triggered PD1 and 10-epi-PD1.

“Maresin” refers to a macrophage-derived mediator of inflammation resolution which is a 12-lipoxygenase-derived metabolite of docosahexaenoic acid (DHA) that are generally have anti-inflammatory, pro-resolving, protective and pro-healing properties. Maresins are members of the specialized pro-resolving mediator class of PUFA metabolites including, for example, maresin 1.

“Specialized pro-resolving mediator” or “SPM” refers to molecular metabolites of poly unsaturated fatty acids (PUFAs). Generally, these molecules are metabolic products formed by a combination of lipoxygenase, cyclooxygenase and cytochrome P450 monooxygenase enzymes. As used herein, the term SPM includes synthetic SPMs that are resistant to being metabolically inactivated. SPMs include, for example, lipoxins, resolvins (e.g., EPA-derived resolvins, DHA-derived resolvins, n-3 DPA-derived resolvins), protectins (e.g., neuroprotectins, DHA-derived protectins/neuroprotectins, n-3 DPA-derived protectins/neuroprotectins), maresins (e.g., DHA-derived maresins, n-3 DPA-derived maresins), n-3 DPA metabolites, N-6 DPA metabolites, oxo-DHA and oxo-DPA metabolites, docosahexaenoyl ethanolamide metabolites, prostaglandins and isoprostanes.

Emerging knowledge and epidemiologic data showed that PUFAs such as EPA, DHA and AA may function in vivo to regulate retinal vaso-obliteration and neovascularization (Kermonant-Duchemin E, et al. Trans-arachidonic acids generated during nitrative stress induce a thrombospondin-1-dependent microvascular degeneration. Nature Med 2005; 11:1339-1345; Seddon J M, Cote J, Rosner B. Progression of Age-Related macular degeneration association with dietary fat, trans-unsaturated fat, nuts, and fish intake. Arch Ophthalmol 2003; 121:1728-1737) and thus, play a role in neovascular age-related macular degeneration, DR, DME and retinopathy of prematurity. Recently, it was noted that increasing tissue levels of EPA/DHA by dietary or genetic manipulation decreased the avascular area of the retina by increasing vessel regrowth after injury, thereby reducing the hypoxic stimulus for neovascularization (Wright M M, Schopfer F J, Baker P R S, Vidyasagar V, Powell P, Chumley P, Iles K E, Freeman B A, Agarwal A. Fatty acid transduction of nitric oxide signaling: Nitrolinoleic acid potently activates endothelial heme oxygenase 1 expression. Proc Natl Acad Sci USA 2006; 103: 4299-4304). Neuroprotectin D1, resolvin D1 and resolvin E1 also protected against neovascularization. These studies indicate that increasing the tissue levels of n-3 fatty acids and/or their products reduce pathological neovascularization and associated retinopathy.

Human retina is very rich in polyunsaturated fatty acids (PUFAs) especially in DHA, EPA, and AA. In the retina, phospholipids account for almost 80% of total lipids and are mainly composed of species belonging to phosphatidylcholine (PC) and phosphatidylethanolamine (PE) subclasses. Within fatty acids esterified on retinal phospholipids, n-3 PUFAs are major components since DHA can represent ˜50% of total fatty acids in the photoreceptor outer segments. DHA is known to play a major role in membrane function in visual process by affecting permeability, fluidity, thickness and the activation of membrane bound proteins. There is evidence to suggest that PUFAs of n-3 series also serve as protective factors in retinal vascular and immuno-regulatory processes, in maintaining the physiologic redox balance, and in cell survival. DHA and its products have the ability to regulate the production and action of VEGF and other angiogenic factors, matrix metalloproteinases, reactive oxygen species, neurotransmitters and pro-inflammatory cytokines.

The composition of free (nonesterified) as well as total (sum of free and esterified) fatty acids (FAs) in human retina is as follows: analysis of free fatty acids (FFAs) revealed that the mean percentage composition of the major components including palmitic acid (PA), stearic acid (SA), oleic acid (OA), AA and DHA were 17.2, 36.7, 15.6, 8.8 and 14.2%, respectively. There were significant correlations between age of the donors' and the content of both free AA and DHA. The mean percentage of total PA, SA, OA, AA and DHA were 22.6, 23.2, 17.7, 11.4 and 21.9%, respectively. There was no association between age and any of the major FAs. The presence of FFAs in the human retina as well as an age-related accumulation of PUFAs suggests an alteration in the metabolism of retinal PUFAs could be due to an increase of oxidative stress and/or decrease of antioxidant defenses during aging.

As noted above, certain embodiments are directed to compositions of bioactive lipids, such as LXA4, resolvins, protectins and/or maresins, and their use for treatment of DR, AMD, DME and retinopathy of prematurity. Without wishing to be bound by theory, it is believed that the selective action of various bioactive lipids and, in particular, to LXA4/resolvins/protectins/maresins can be attributed to one or more than one of the following events: (i) potent inhibitors of VEGF production and action (LXA4>resolvin D1=resolvin E1>protectin D1>maresin); (ii) inhibit PGE2 synthesis and antagonized to pro-inflammatory actions of leukotriene D4 (LTD4). Both PGE2 and LTD4 have pro-inflammatory actions and enhance angiogenesis; (iii) LXA4/resolvins/protectins/maresins inhibit angiogenesis that is an important feature of DR; (iv) LXA4/resolvins/protectins/maresins prevent neutrophils and macrophages accumulation and activation in DR (Kakehashi et al. Diabetes Res Clin Pract 2008; 79: 438-445; Moreno et al. J Immunol 2013; 191: 6136-6146); (v) LXA4/resolvins/protectins/maresins inhibit the production of IL-17 that is capable of inducing resistance to anti-VEGF therapies and thus, indirectly enhance anti-angiogenic action of anti-VEGF therapy (Diaz-Gerevini et al. Nutrition 2016; 32: 174-178); (vi) LXA4/resolvins/protectins/maresins have anti-inflammatory actions and thus, possess anti-angiogenic actions; (vii) LXA4/resolvins/protectins/maresins have direct anti-VEGF action and thus, decrease angiogenic processes seen in DR; (viii) LXA4/resolvins/protectins/maresins inhibit vascular endothelial cell proliferation and migration that are essential for angiogenesis seen in DR; (ix) studies performed in support of embodiments of the present invention revealed that LXA4/resolvins/protectins/maresins interact with each other and potentiate and mediate each other's action (Lee et al. Int J Biochem Cell Biol 2013; 45: 2801-2807) implying that these bioactive lipids need to be administered together and in specific proportion so that their beneficial actions are optimized; (x) LXA4/resolvins/protectins/maresins inhibit tube formation from human umbilical vein endothelial cells (HUVECs) stimulated with VEGF and LTD4 (Baker et al. J Immunol 2009; 182:3819-3826); (xi) LXA4/resolvins/protectins/maresins inhibit VEGF-induced phosphorylation of PLC-γ1, ERK1/2, and Akt that are needed for increasing intracellular levels of inositol 1,4,5-trisphosphate and elevation of intracellular calcium, as well as activation of the MAPKs that leads to proliferation of endothelial cells and survival of endothelial cells and thus, these bioactive lipids suppress pathological angiogenesis; (xii) LXA4/resolvins/protectins/maresins decrease pro-inflammatory cytokines: IL-6, TNF-α, IL-8, and IFN-γ and up-regulate IL-10 production that leads to the suppression of pro-inflammatory process that initiates and perpetuates pathological angiogenesis seen in DR; (xiii) LXA4/resolvins/protectins/maresins stimulate actin rearrangement and inhibit VEGF- and LTD4-stimulated angiogenesis of HUVECs that underlie the pathobiology of pathological angiogenesis; (xiv) LXA4/resolvins/protectins/maresins inhibit endothelial cell chemotaxis initiated by VEGF, a process that is needed for pathological angiogenesis seen in DR; (xv) platelet/endothelial cell adhesion molecule-1 (or CD31), a member of the Ig superfamily that is strongly expressed at the endothelial cell-cell junction, is present on platelets as well as leukocytes, and is held to play a role in angiogenesis and in transendothelial migration of leukocytes is stimulated by VEGF and this VEGF-induced increase in the expression of CD31 is inhibited by LXA4/resolvins/protectins/maresins that explains the anti-angiogenic action of these bioactive lipids in pathological angiogenesis seen in DR (Yang F, Xie J, Wang W, Xie Y, Sun H, Jin Y, Xu D, Chen B, Andersson R, Zhou M. PLoS One 2014; 9: e108525); (xv) COX-2 expression and prostaglandin E2 (PGE2) levels, and expression of PGE2 receptors EP-2 and EP-4 were upregulated with chronic inflammation that correlated with increased corneal PGE2 formation and marked neovascularization in an animal model of chronic inflammation induced by corneal suture method. On the other hand, acute abrasion injury that produces acute but self-limiting corneal injury did not produce alterations in PGE2 levels or EP expression. It was noted that PGE2 treatment amplified PMN infiltration and the angiogenic response to chronic inflammation but did not affect wound healing or PMN infiltration. Interestingly, exacerbated inflammatory neovascularization with PGE2 treatment was found to be independent of the VEGF circuit but was associated with a significant induction of the eotaxin-CCR3 axis. These findings suggest that corneal and probably even retinal (and other tissues) PGE2 mediate inflammatory neovascularization that is independent of VEGF (Liclican E L, Nguyen V, Sullivan A B, Gronert K. Selective activation of the prostaglandin E2 circuit in chronic injury-induced pathologic angiogenesis. Invest Ophthalmol Vis Sci 2010; 51: 6311-6320). Since VEGF is a potent inducer of PGE2 production, it is likely that PGE2 is the mediator of VEGF-induced neovascularization and pathological angiogenesis (Tamura K, Sakurai T, Kogo H. Relationship between prostaglandin E2 and vascular endothelial growth factor (VEGF) in angiogenesis in human vascular endothelial cells. Vascul Pharmacol 2006; 44: 411-416; Wu G, Mannam A P, Wu J, Kirbis S, Shie J L, Chen C, Laham R J, Sellke F W, Li J. Hypoxia induces myocyte-dependent COX-2 regulation in endothelial cells: role of VEGF. Am J Physiol Heart Circ Physiol 2003; 285: H2420-H2429). In addition, PGE2 is also a potent inducer of VEGF production (Cheng T, Cao W, Wen R, Steinberg R H, LaVail M M. Prostaglandin E2 induces vascular endothelial growth factor and basic fibroblast growth factor mRNA expression in cultured rat Müller cells. Invest Ophthalmol Vis Sci 1998; 39: 581-591; Yanni S E, McCollum G W, Penn J S. Genetic deletion of COX-2 diminishes VEGF production in mouse retinal Müller cells. Exp Eye Res 2010; 91: 34-41; Mulligan J K, Rosenzweig S A, Young M R. Tumor secretion of VEGF induces endothelial cells to suppress T cell functions through the production of PGE2. J Immunother 2010; 33: 126-135). Thus, both PGE2 and VEGF participate in neovascularization and pathological angiogenesis. This implies that inhibition of either PGE2 or VEGF can suppress neovascularization and pathological angiogenesis. Since both PGE2 and VEGF interact with each other and one enhances the production of the other (PGE2 enhances VEGF production and VEGF enhances PGE2 production), suppression of either PGE2 or VEGF alone is not sufficient to suppress neovascularization and pathological angiogenesis. Inhibition of both PGE2 and VEGF is desirable to induce significant suppression of neovascularization and pathological angiogenesis. In this context, bioactive lipids (lipoxins, resolvins, protectins and maresins) score over conventional COX2 inhibitors (that suppress only PGE2 production) and corticosteroids (that suppress COX-2 and also of delta 6 and delta 5 desaturases and thus, induce deficiency of AA, EPA and DHA), and anti-VEGF antibodies that neutralize only VEGF since, they effectively suppress both PGE2 and VEGF production. Thus, bioactive lipids are far superior in inhibiting neovascularization and pathological angiogenesis and are effective in the management of DR, AMD, DME and retinopathy of prematurity. (xvi) One important feature of DR is an increase in the apoptotic death of retinal capillary pericytes and endothelial cells that are considered as the initiating events in the development of DR. The inventor showed previously that lipoxins, resolvins, protectins and maresins (lipoxins>resolvins>protectins>maresins) have cytoprotective actions. In an in vitro study, it was noted that these bioactive lipids prevented cytotoxic action of chemicals such as alloxan and streptozotocin on RINF cells (insulin producing pancreatic β cells) and in vivo. In addition, these bioactive lipids enhanced the production of brain-derived neurotropic factor (BDNF), a neurotrophin that is known to protect neuronal cells from endogenous and exogenous toxins. Thus, bioactive lipids and BDNF when present in adequate amounts can prevent apoptotic death of retinal capillary pericytes and endothelial cells and prevent DR, DME, AMD and retinopathy of prematurity. In addition, BDNF also enhanced the production of bioactive lipids especially that of LXA4. Thus, bioactive lipids and BDNF are able to enhance each other's production to protect retinal capillary pericytes and endothelial cells to prevent DR, DME, AMD and retinopathy of prematurity. (xvii) In addition to their anti-inflammatory and cytoprotective actions, these bioactive lipids also have the ability to act on stem cells and regulate their survival, proliferation and differentiation. Thus, these bioactive lipids not only arrest and progression of DR, DME, AMD and retinopathy of prematurity but induce stem cell proliferation and differentiation such that retinal capillary pericytes and endothelial cells, and retinal pigment epithelial cells are regenerated and replace them effectively to restore retinal architecture to normal. Thus, these bioactive lipids are able to restore normal retinal architecture by tissue regeneration. Though all bioactive lipids have this tissue regenerating capacity, maresins seem to be the most potent (maresins>protectins≥resolvins≥lipoxins). (xviii) There is evidence to suggest that oxidative stress (Bansal S, Chawla D, Siddarth M, Banerjee B D, Madhu S V, Tripathi A K. A study on serum advanced glycation end products and its association with oxidative stress and paraoxonase activity in type 2 diabetic patients with vascular complications. Clin Biochem 2013; 46: 109-114; Mandal L K, Choudhuri S, Dutta D, et al. Oxidative stress-associated neuroretinal dysfunction and nitrosative stress in diabetic retinopathy. Can J Diabetes 2013; 37: 401-407); retinal vascular endothelial dysfunction (Hein T W, Potts L B, Xu W, Yuen J Z, Kuo L. Temporal development of retinal arteriolar endothelial dysfunction in porcine type 1 diabetes. Invest Ophthalmol Vis Sci 2012; 53: 7943-7949); and consequent increased vascular permeability (Othman A, Ahmad S, Megyerdi S, et al. 12/15-Lipoxygenase-derived lipid metabolites induce retinal endothelial cell barrier dysfunction: contribution of NADPH oxidase. PLoS One 2013; 8: e57254); enhanced expression of adhesion molecules (Noda K, Nakao S, Ishida S, Ishibashi T. Leukocyte adhesion molecules in diabetic retinopathy. J Ophthalmol 2012; 2012: 279037; Gustaysson C, Agardh C D, Zetterqvist A V, Nilsson J, Agardh E, Gomez M F. Vascular cellular adhesion molecule-1 (VCAM-1) expression in mice retinal vessels is affected by both hyperglycemia and hyperlipidemia. PloS One 2010; 5: e12699); and increased production and action of pro-inflammatory cytokines play a significant role (Myśliwiec M, Balcerska A, Zorena K, Myśliwska J, Lipowski P, Raczyńska K. The role of vascular endothelial growth factor, tumor necrosis factor alpha and interleukin-6 in pathogenesis of diabetic retinopathy. Diabetes Res Clin Pract 2008; 79: 141-146; Hernández C, Segura R M, Fonollosa A, Carrasco E, Francisco G, Simo R. Interleukin-8, monocyte chemoattractant protein-1 and IL-10 in the vitreous fluid of patients with proliferative diabetic retinopathy. Diabet Med 2005; 22: 719-722) suggesting that DR is a low-grade inflammatory condition. This is further supported by the presence of inflammatory signs in DR that are more at the microscopic level. The features of inflammation seen in DR include vessel dilatation, altered flow, exudation of fluids including plasma proteins, and leucocyte accumulation and migration (Adamis A P. Is diabetic retinopathy an inflammatory disease? Br J Ophthalmol 2002; 86: 363-365). These local microscopic signs of inflammation in DR are due to increased production of tumor necrosis factor-α (TNF-α), VEGF, prostaglandins (PGs), enhanced expression of intercellular adhesion molecule-1 (ICAM-1) on the vasculature, β2 integrins on the leucocytes, and vascular cell adhesion molecule-1 (VCAM-1) and VLA-4 (very late antigen-4, also called integrin α4β1) (Myśliwiec M, Balcerska A, Zorena K, Myśliwska J, Lipowski P, Raczyńska K. The role of vascular endothelial growth factor, tumor necrosis factor alpha and interleukin-6 in pathogenesis of diabetic retinopathy. Diabetes Res Clin Pract 2008; 79: 141-146; Hernández C, Segura R M, Fonollosa A, Carrasco E, Francisco G, Simó R. Interleukin-8, monocyte chemoattractant protein-1 and IL-10 in the vitreous fluid of patients with proliferative diabetic retinopathy. Diabet Med 2005; 22: 719-722; Miyamoto K, Khosrof S, Bursell S E, et al. Prevention of leukostasis and vascular leakage in streptozotocin-induced diabetic retinopathy via intercellular adhesion molecule-1 inhibition. Proc Natl Acad Sci USA 1999; 96: 10836-10841; Canas-Barouch F, Miyamoto K, Allport J R, et al. Integrin-mediated neutrophil adhesion and retinal leukostasis in diabetes. Invest Ophthalmol Vis Sci 2000; 41:1153-1158; Schoenberger S D, Kim S J, Sheng J, Rezaei K A, Lalezary M, Cherney E. Increased prostaglandin E2 (PGE2) levels in proliferative diabetic retinopathy, and correlation with VEGF and inflammatory cytokines. Invest Ophthalmol Vis Sci 2012; 53: 5906-5911). These events enhance leukocyte adherence and accumulation within the vasculature of the retina (Miyamoto K, Hiroshiba N, Tsujikawa A, Ogura Y. In vivo demonstration of increased leukocyte entrapment in retinal microcirculation of diabetic rats. Invest Ophthalmol Vis Sci 1998; 39: 2190-2194), which may precede the occurrence of DR. The leukocyte adherence and migration lead to vascular dysfunction as a result of increased production of reactive oxygen species (ROS) and lipid peroxidation that occurs locally, which results in a subtle breakdown of the blood-retinal barrier, premature endothelial cell injury and death, and capillary ischaemia/reperfusion (Joussen A M, Murata T, Tsujikawa A, Kirchhof B, Bursell S E, Adamis A P. Leukocyte-mediated endothelial cell injury and death in the diabetic retina. Am J Pathol 2001; 158: 147-152). Bioactive lipids (LXA4>resolvins≥protectins≥maresins) inhibit free radical generation, lipid peroxidation and suppress production of inflammatory cytokines, proinflammatory PGE2, inhibit leukocyte migration and adherence to vascular endothelial cells, and restore retinal vascular endothelial function and retinal endothelial cell barrier function thus, prevent or even reverse DR, AMD, DME and retinopathy of prematurity.

Without wishing to be bound by theory, it is believed that bioactive lipids (lipoxins, resolvins, protectins and maresins): (i) inhibit abnormal angiogenesis; (ii) protect retinal capillary pericytes and endothelial cells, and retinal pigment epithelial cells; (iii) suppress the production of proinflammatory cytokines: IL-6 and TNF-α and thus, suppress inflammation; (iv) inhibit leukocyte migration and activation and adherence to vascular endothelial cells; (v) suppress the production of PGE2, a pro-inflammatory molecule; (vi) inhibit VEGF and free radical generation and (vii) at the same time enhance the production of BDNF, a neuroprotective molecule, that ultimately leads to inhibition, progression and even reversal of DR, DME, AMD and retinopathy of prematurity.

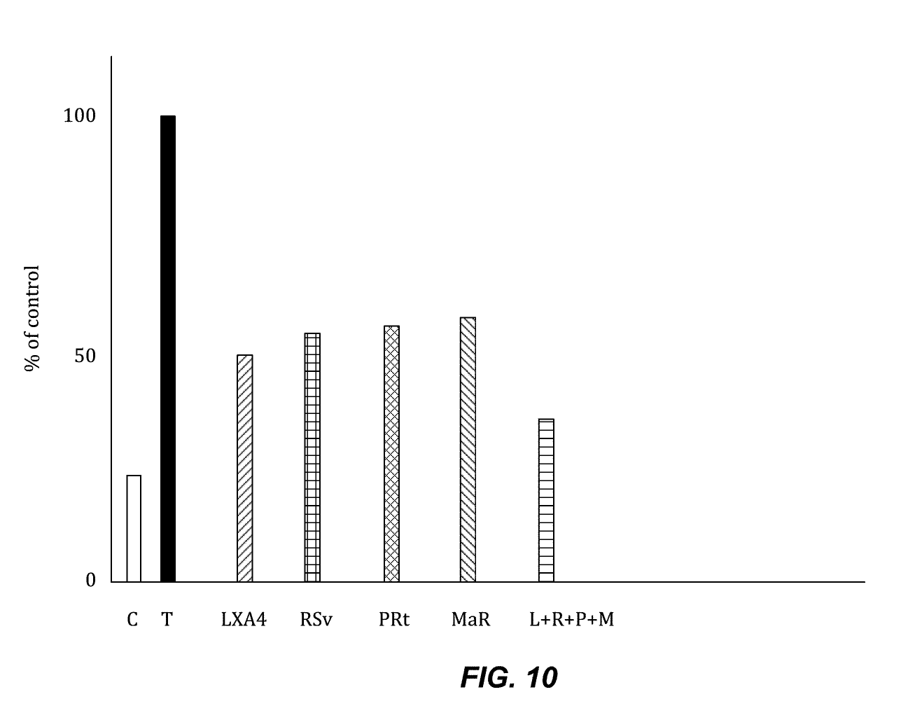

In order to verify the above mentioned possibilities, the inventor studied the effect of LXA4, resolvins, protectins and maresins on (i) angiogenesis, (ii) their cytoprotective action, (iii) production of IL-6 and TNF-α, PGE2 and VEGF, (iv) leukocyte migration and adherence to vascular endothelial cells and (v) production of BDNF and these results are shown in FIGS. 10-15.

There are several advantages of LXA4/resolvins/protectins/maresins (individually or in combination) treatment as described in certain embodiments of the invention. In some embodiments, a single injection per day for 7 to 10 days (of LXA4/Resolvins/protectins/maresins or in combination),at separate times is adequate to produce almost permanent regression of DR, AMD, DME and retinopathy of prematurity by suppressing angiogenesis/neoangiogenesis with no or very little recurrence of the new blood vessel formation. LXA4/resolvins/protectins/maresins or in combination and their salts are non-antigenic, are known to be relatively safe and stable in the dosages described herein.

Without wishing to be bound by theory, certain aspects of some embodiments are believed to relate, at least in part, to the discovery of the novel and highly beneficial action of LXA4/resolvins/protectins/maresins induced regression of pathological angiogenesis and/or prevented further formation of new blood vessels. In some embodiments, this effect is particularly observed when the bioactive lipids, such as LXA4, are administered directly into the vitreal cavity (e.g., injected intravitreally). It is believed that the selective anti-angiogenic action of LXA4/resolvins/protectins/maresins administered is due to the anti-inflammatory and anti-angiogenic action of these bioactive lipids only that are abnormal but not of the normal blood vessels or normal retinal cells or retinal vascular endothelial cells or retinal pigment epithelial cells.

All bioactive lipids and in particular, lipoxins, resolvins, protectins and maresins are not water soluble as a result of which it is difficult to deliver to vitreal cavity and to act on retinal cells that are very rich in lipids and so may not get solubilized in the vitreal fluid for their subsequent integration to retinal and other cell membranes that are rich in lipids. Solvents that are used to dissolve bioactive lipids have biological actions that could render lipoxins, resolvins, protectins and maresins inactive or interfere with their beneficial actions. For example, DMSO (dimethyl sulfoxide) is a solvent that could be used to dissolve various bioactive lipids. But, DMSO is a toxic substance (Rubin L F, Mattis P A. Dimethyl sulfoxide: lens changes in dogs during oral administration. Science 1966; 153: 83-84; Noel P R, et al. The toxicity of dimethyl sulphoxide (DMSO) for the dog, pig, rat and rabbit. Toxicology 1975; 3: 143-169).

Similarly, bioactive lipids are soluble in other lipid solvents such as ethyl acetate, methanol, chloroform, acetone, hexane, isopropanol, methyl-tert-butyl ether (MTBE), or detergent such as Triton X-114. But, all these solvents themselves have potent cytotoxic actions and thus, bioactive lipids dissolved in these solvents show toxic action on normal cells including retinal cells, retinal vascular endothelial cells, retinal capillary pericytes, endothelial cells, and retinal pigment epithelial cells. Hence are not suitable to use them as delivery vehicles of bioactive lipids.

Certain embodiments provide a composition comprising one or more bioactive lipid, salt or derivative thereof, the bioactive lipid selected from the group consisting of a lipoxin, a resolvin, a protectin, and a maresin, a stabilizing agent, a solution selected from the group consisting of saline and phosphate buffered saline, and ethanol;

wherein the concentration of the stabilizing agent and ethanol does not interfere with the anti-inflammatory, immunomodulatory and anti-angiogenic activity of the bioactive lipid.

One embodiment provides a composition comprising one or more bioactive lipid, salt or derivative thereof, wherein the bioactive lipid is a specialized pro-resolving mediator (SPM), a stabilizing agent, a solution selected from the group consisting of saline and phosphate buffered saline and ethanol;

wherein the concentration of the stabilizing agent and ethanol does not interfere with the anti-inflammatory, immunomodulatory and anti-angiogenic activity of the bioactive lipid. In some of those embodiments, the SPM is synthetic SPM, for example, a synthetic SPM that is resistant to metabolic inactivation.

In view of this solubility issue and non-availability of a suitable non-toxic solvent to dissolve various bioactive lipids for their appropriate delivery to human tissues including retina and surrounding tissues, especially into the vitreal cavity, no progress has been made in the parenteral delivery of various bioactive lipids for various human diseases including DR, DME, AMD and retinopathy of prematurity. Identification and/or development of a suitable solvent for bioactive lipids (since in the absence of a suitable solvent bioactive lipids cannot be injected into human vitreal cavity) is needed so that its anti-angiogenic, anti-inflammatory and cytoprotective actions can be exploited in an appropriate fashion for the treatment of DR, AMD, DME and retinopathy of prematurity. This is so since, if the solvent used to dissolve bioactive lipids is toxic then it is likely to produce significant side effects especially when these lipids are injected into the vitreal cavity of the eye. Hence, a suitable solvent should be identified or developed to dissolve bioactive lipids such that the solvent is non-toxic and delivery of bioactive lipids can be performed safely. Furthermore, the solvent used should make bioactive lipids, at least, partially water soluble so that further dilution of the solution is possible to deliver the required amount(s) of the bioactive lipids.

In the absence of such a suitable water soluble or at least partially water soluble solvent system for dissolving and delivering bioactive lipids, one will not be able to deliver appropriate amounts of bioactive lipids needed to produce the desired actions. In the absence of such a suitable water soluble or at least partially water soluble system, the amount(s) of bioactive lipids delivered to the tissues will be either too high or too low but not appropriate. Thus, development of a suitable solvent system for the delivery of bioactive lipids is needed so that even if accidentally bioactive lipids are injected into vitreal cavity no side effects will occur due to the solvent used for delivering bioactive lipids. This is important since, bioactive lipids by themselves are not toxic to normal cells and to rule out the possibility that the side effects observed are due to the solvent system used for the delivery of bioactive lipids. Thus, in certain embodiments of the composition, the concentration of ethanol ranges from 0.0001% to 0.01%, for example, from 0.0005% to 0.009%, from 0.001% to 0.0075%, from 0.005% to 0.0075%, from 0.006% to 0.0075% w/w, w/v or v/v.

Without wishing to be bound by theory, it is believed that, in certain embodiments, there is an interaction between the bioactive lipids and the solvent system used to dissolve it which may account for the effectiveness of the treatment. Thus, the newly discovered solvent system (and method for preparation of the composition) used which, in certain embodiments, comprises pure ethyl alcohol and a stabilizing agent {small amounts of albumin pg to mg/μg to gram of bioactive lipids} is believed to synergistically interact with the bioactive lipids to produce a therapeutic effect which is unexpectedly different than the effect of either bioactive lipids or the solvent and/or agent/stabilizing agent alone. In some embodiments, the stabilizing agent is human albumin. In addition, the presence of small amounts of human albumin is essential to stabilize bioactive lipids such that bioactive lipids are able to bring about their beneficial actions in order to prevent, reverse and decrease DR, AMD, DME and retinopathy of prematurity.

The optional stabilizing agent can be present in various amounts. For example, in some embodiments the optional stabilizing agent is present in the composition in amounts ranging from in about 1 picogram/gram of bioactive lipid(s) to about 10 micrograms/g ram of bioactive lipid(s). In some embodiments the optional stabilizing agent is present in the composition in amounts ranging from 1 picogram/gram of bioactive lipid(s) to about 100 nanograms/gram of bioactive lipid(s). In other embodiments, the concentration of stabilizing agent in the composition ranges from about 1 picogram/gram of bioactive lipid(s) to about 10 nanograms/gram of bioactive lipid(s). In other embodiments, the concentration of stabilizing agent in the composition ranges from about 1 picogram/gram of bioactive lipid(s) to about 1 nanograms/gram of bioactive lipid(s). In other embodiments, the concentration of stabilizing agent in the composition ranges from about 1 picogram/gram of bioactive lipid(s) to about 100 picograms/gram of bioactive lipid(s). In other embodiments, the concentration of stabilizing agent in the composition ranges from about 1 picogram/gram of bioactive lipid(s) to about 10 picograms/gram of bioactive lipid(s). In certain specific embodiments, the concentration of the stabilizing agent ranges from 1 pg/gram to about 10 μg/gram of bioactive lipid, for example from 5 pg/gram to about 5 μg/gram, or from about 10 pg/gram to about 1 μg/gram.

Accordingly, some embodiments provide a composition comprising a bioactive lipid, such as lipoxin A4, lipoxin B4, resolvin D1, resolvin E1, protectin D1, and/or maresins, ethyl alcohol and an optional stabilizing reagent. Thus, in certain embodiments, the bioactive lipid is selected from the group consisting of lipoxin A4, lipoxin B4, resolvin D1, resolvin E1, protectin D1, and maresin 1. In more specific embodiments, the composition comprises a lipoxin, a resolvin, a protectin, and a maresin in a ratio ranging from 0.5:1:1:1 to 2:1:1:1, from 1:0.5:1:1 to 1:2:1:1, from 1:1:0.5:1 to 1:1:2:1, from 1:1:1:0.5 to 1:1:1:2, from 0.5:0.5:1:1 to 2:2:1:1, from 1:1:0.5:0.5 to 1:1:2:2, from 0.5:1:0.5:1 to 2:1:2:1, from 1:0.5:1:0.5 to 1:2:1:2, from 0.5:1:1:0.5 to 2:1:1:2, respectively, for example, 1:1:1:1, respectively. In some embodiments, the composition comprises lipoxin A4, resolvin E1, protectin D1, and maresin 1 in a ratio of 0.5:1:1:1 to 2:1:1:1, from 1:0.5:1:1 to 1:2:1:1, from 1:1:0.5:1 to 1:1:2:1, from 1:1:1:0.5 to 1:1:1:2, from 0.5:0.5:1:1 to 2:2:1:1, from 1:1:0.5:0.5 to 1:1:2:2, from 0.5:1:0.5:1 to 2:1:2:1, from 1:0.5:1:0.5 to 1:2:1:2, from 0.5:1:1:0.5 to 2:1:1:2, respectively, for example, 1:1:1:1, respectively. In some embodiments, the bioactive lipid is lipoxin A4. The present inventor unexpectedly discovered that when the ethyl alcohol concentration is outside the optimal range in any solution wherein bioactive lipids are present, the antiangiogenic, antiinflammatory and cytoprotective actions of bioactive lipids are suboptimal and the bioactive lipids unstable. This is an unexpected observation since it is not typically expected that any solvent used for dissolving an active chemical would be so critical for the stability and activity of the active chemical. However, in the case of bioactive lipids, this was found to be true.

Thus, as already discussed above, the surprising and novel observation of the inventor is the fact that any deviation in the solvent content outside the optimal range in a given solution in which bioactive lipids are present, the activity, stability and their beneficial actions were altered such that bioactive lipids became relatively inactive, unstable and their antiinflammatory, antiangiogenic and cytoprotective actions inefficient. In general, it is believed that two or more molecules or drugs or compounds that have similar properties (with similar or different mechanisms of action(s)) will enhance the final action of each other. For instance, in cancer therapy two or more drugs with different mechanism(s) of action are employed so that tumor cells are killed to give relief to the patient. Thus, use of more than two or three drugs in different combinations is often used in cancer chemotherapy. Based on similar principle, it has been proposed that a combination of anti-angiogenic molecules: endostatin and angiostatin when combined with salts of bioactive lipids will potentiate each other's action especially when given intra-vitreally to prevent neoangiogenesis (see U.S. Pat. No. 6,380,253). Though this argument looks reasonable, to the surprise of the inventor it was found that this is not the case.

The inventor unexpectedly discovered that the free acid form of bioactive lipids are more potent than sodium salt, ethyl ester, methyl ester or other forms of salts, esters, glycerides, amides, or phospholipids, or alkylated, alkoxylated, halogenated, sulfonated, or phosphorylated forms of bioactive. This is despite the fact that such derivatives of bioactive lipids (namely: sodium salt, ethyl ester, methyl ester or other forms of salts, esters, glycerides, amides, or phospholipids, or alkylated, alkoxylated, halogenated, sulfonated, or phosphorylated forms) are more stable. For some unexplained reason such derivatives of bioactive lipids (namely: sodium salt, ethyl ester, methyl ester or other forms of salts, esters, glycerides, amides, or phospholipids, or alkylated, alkoxylated, halogenated, sulfonated, or phosphorylated forms) are less active than the free acid forms. Thus, in certain embodiments, the bioactive lipid comprises a bioactive lipid in the free acid form.

In general, it was believed that the potency of actions of a free acid form is equal to or similar to sodium salt, ethyl ester, methyl ester or other forms of salts, esters, glycerides, amides, or phospholipids, or alkylated, alkoxylated, halogenated, sulfonated, or phosphorylated forms of the acid form. However, it was unexpectedly discovered that the free acid form of bioactive lipids are the most potent in bringing about their anti-angiogenic, antiinflammatory and cytoprotective actions in comparison to sodium salt, ethyl and methyl esters and other forms of bioactive lipids. Surprisingly it was also observed that several chemically synthesized stable analogues of bioactive lipids (for example see patent no. CA2466418) were less effective compared to the naturally occurring endogenous bioactive lipids though the synthetic analogues are apparently more stable.

In one aspect, one embodiment provides a method of preparation of bioactive lipids such that they are made more water soluble, more stable at room temperature and are more active than the methyl, ethyl, or sodium and other types of salts of bioactive lipids such that they are able to get incorporated into the membrane of retinal capillary pericytes, endothelial cells, and retinal pigment epithelial cells better and bring about their beneficial antiangiogenic, antiinflammatory and cytoprotective actions in a desirable fashion is presented. Some embodiments also provide methods of selectively causing anti-angiogenic, antiinflammatory and cytoprotective action in patients with DR, AMD, DME and retinopathy of prematurity, with the result that new blood vessels and collaterals are not formed to sustain pathological retinopathy.

In this context, embodiments of the present invention provides methods for selectively reducing

(i) the growth and inducing apoptosis of endothelial cells that form abnormal tube like structures that are precursors of pathological angiogenic vessels;

(ii) inhibiting the production of angiogenic factors including VEGF;

(iii) blocking PGE2 production;

(iv) preventing angiogenesis;

(v) suppressing inflammation locally;

(ix) enhancing the expression of p53;

(vi) altering the expression of Bcl-2 and BAX; and

(xi) increasing the production of LXA4 in an autocrine fashion the surrounding normal cells when bioactive lipids are injected in to the vitreal cavity.

In one embodiment, methods in which a therapeutically effective amount of a solution of bioactive lipids is injected, thereby selectively reducing the abnormal angiogenesis, suppressing unwanted inflammation and protecting retinal capillary pericytes, endothelial cells, and retinal pigment epithelial cells occurs is provided. In preferred embodiments, the amount of bioactive lipids present in the administered solution is sufficient not only to inhibit abnormal angiogenesis and suppress inflammation but also to decrease the production of angiogenic factor including VEGF by the human retinal pigment epithelial cells and choroid and the surrounding cells starting in a period of about 24 hours. In preferred embodiments, the therapeutically effective amount of bioactive lipids is between 1.0 ng to 100 mg per day, most preferable between 10.0 μg to 500 μg per day irrespective of the degree or severity of DR, AMD, DME and retinopathy of prematurity.

In certain embodiments disclosed herein, the administering results in at least one of the following:

i) selectively reducing the growth and inducing the apoptosis of endothelial cells that form abnormal tube-like structures, which are precursors of pathological angiogenic vessels;

ii) inhibiting the production of angiogenic factors including VEGF;

iii) blocking PGE2 production;

iv) preventing angiogenesis, including inhibiting the growth of new blood vessels;

v) suppressing inflammation locally;

vi) enhancing expression of p53;

vii) altering the expression of Bcl-2 and BAX;

viii) increasing production of lipoxin A4; or

ix) reducing abnormal angiogenesis.

In some embodiments, bioactive lipids are in the form of a free acid and is made soluble in water using a distinct method of solubilizing it without losing its activity (since normally bioactive lipids become inactive rapidly when present in water) using a stabilizing agent that not only makes bioactive lipids stable but does not interfere with their action(s) and injecting in to the vitreal cavity of eye.

In some embodiments, in addition to injecting bioactive lipids into the vitreal cavity of the eye, the following measurements or investigations are done that include:

(i) a fluorescent angiogram;

(ii) direct and indirect optic fundal examination of the eye;

(iii) optical coherence tomography (OCT) exam that provides cross-sectional images of the retina that shows the thickness of the retina, which will help determine whether fluid has leaked into retinal tissue;

(iv) specifically measuring central retinal thickness (CRT), and

(v) best-corrected visual acuity (BCVA) to monitor how treatment is working. These examinations (i to v) are taken and recorded before and after the injection(s). That is, exams are taken every 4 weeks, most preferably 24 hours after 7 days or 10 days of injection or administration of bioactive lipids into the vitreal cavity tumor mass to assess and record the extent of remission of DR, DME, AMD and retinopathy of prematurity. In some embodiments, administering comprises a single injection repeated at an interval ranging from 1 day to 6 weeks and continued for a period ranging from 4 weeks to 5 years.

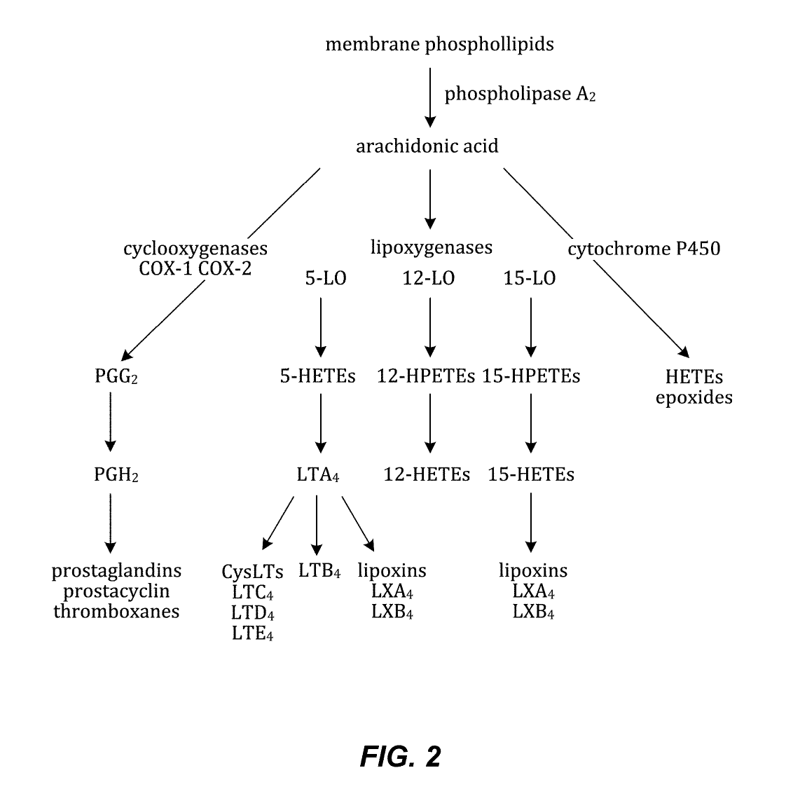

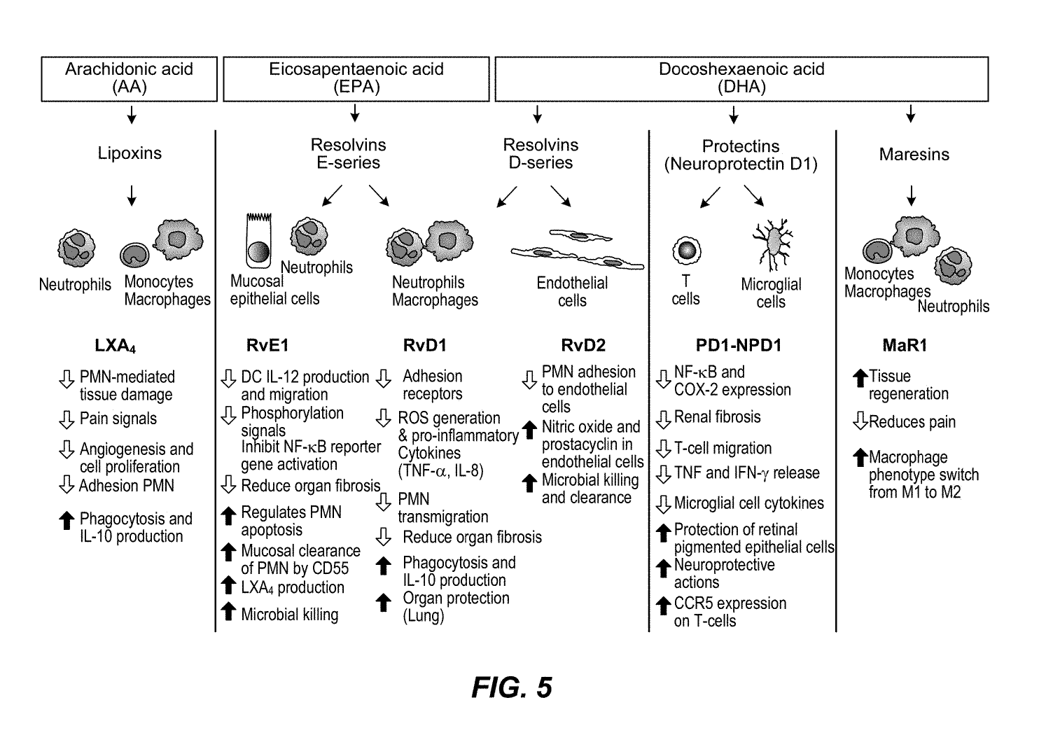

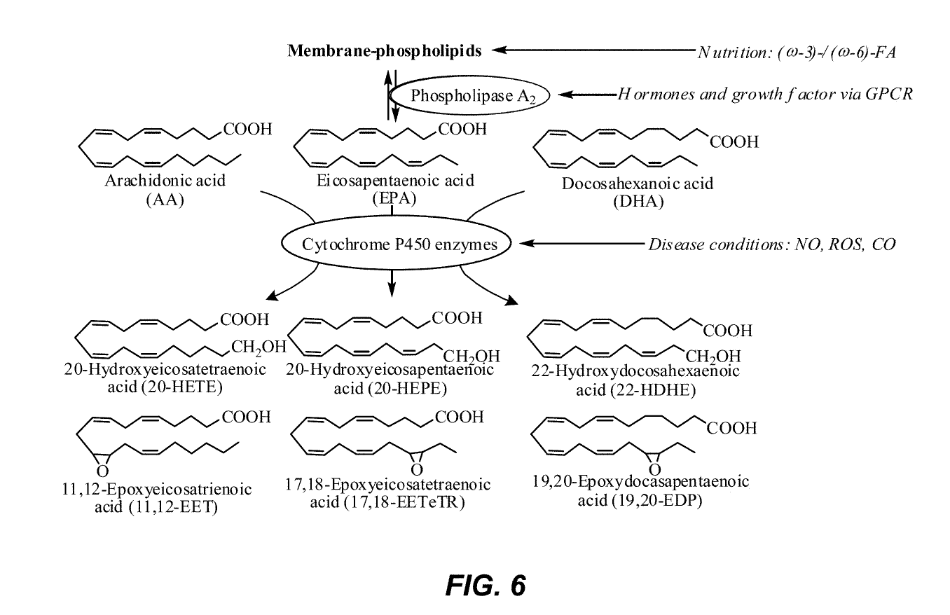

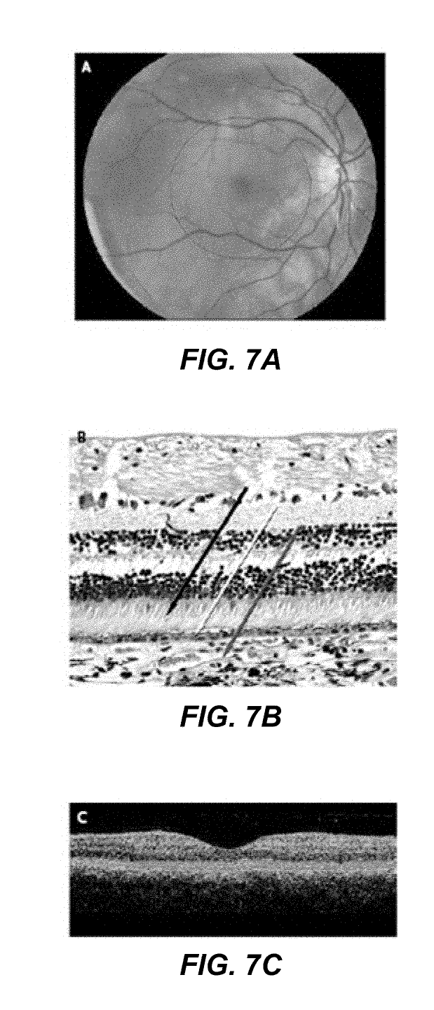

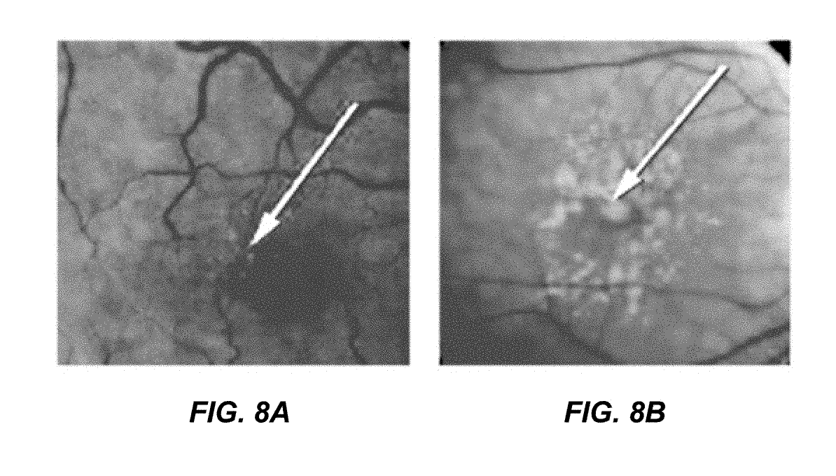

In some embodiments, the injected bioactive lipid could be a lipoxin A4, resolvin, protectin and/or maresin. These bioactive lipids could any one of lipoxin, resolvin, protectin and/or maresin. In certain preferred embodiments, the bioactive lipid is selected from lipoxins, resolvins, protectins and maresins (see FIGS. 2-5 for various bioactive lipids).

In some embodiments, the bioactive lipid comprises a bioactive lipid salt, for example, sodium salt, a magnesium salt, a manganese salt, an iron salt, a copper salt, an iodide salt, or combinations thereof In other embodiments, the bioactive lipid comprises a bioactive lipid derivative, for example, a glyceride, an ester, an ether, an amide, a phospholipid, an alkylated lipid, an alkoxylated lipid, a halogenated lipid, a sulfonated lipid, a phosphorylated lipid, or combinations thereof. In some embodiments, the bioactive lipid is administered in the form of free acid, or a salt, such as a sodium salt, magnesium salt, a manganese salt, an iron salt, a copper salt, or an iodide salt. In some preferred embodiments, the bioactive lipid is in the form of a fatty acid derivative, such as a glyceride, ester, ether, amide, or phospholipid, or an alkylated, alkoxylated, halogenated, sulfonated, or phosphorylated form of the fatty acid.

In some embodiments, the pathological retinopathy is due to DR, DME, AMD, and retinopathy of prematurity.

Accordingly, in one embodiment is provided a pharmaceutical composition comprising: a

bioactive lipid in the free acid form;
saline or phosphate buffered saline; and
from 0.01% to 0.0001% ethanol.

In some embodiments, the bioactive lipid is selected from the group consisting of lipoxin A4, lipoxin B4, resolvin E1, resolvin E2, resolvin D1, resolvin D2, protectin D1, protectin D2, and maresin 1. For example is certain embodiment the bioactive lipid is lipoxin A4.

In some embodiments, in addition to the bioactive lipid (lipoxins, resolvins, protectins and maresins), a therapeutically effective amount of a compound selected from anti-tumor necrosis factor, anti-VEGF, and anti-EGF polyclonal or monoclonal antibodies and corticosteroid is injected in combination with bioactive lipids.

In other embodiments, the bioactive lipid is covalently conjugated with a pharmaceutical agent chosen from anti-TNF, anti-VEGF, and anti-EGF polyclonal or monoclonal antibody.

In another embodiment, pharmaceutical compositions of bioactive lipid(s), or free acid or salt of bioactive lipid, in combination with a corticosteroid molecule are provided.

In one embodiment, a method of preparation of bioactive lipid(s) (e.g., in free acid form) such that it is made more water soluble, more stable at room temperature and is more active than the methyl, ethyl, or sodium and other types of salts of bioactive lipid such that it is able to enter the cell membrane of retinal capillary pericytes, endothelial cells, and retinal pigment epithelial cells, choroid cells and the surrounding cells better and bring about their selective antiangiogenic, antiinflammatory and cytoprotective action in a desirable fashion is provided. All bioactive lipids and in particular, lipoxin A4/protectins/resolvins/maresins are not water soluble as a result of which it is difficult to deliver to various cells. This is so since, solvents that are used to dissolve bioactive lipids and, in particular lipoxin A4/resolvin/protectin/maresin have biological actions that could render these bioactive lipids inactive or interfere with their beneficial actions especially, antiangiogenic action.

For example, DMSO (dimethyl sulfoxide) is a solvent that could be used to dissolve various bioactive lipids (lipoxins, resolvins, protectins and maresins). But, DMSO is a toxic chemical that may prove to be harmful to the delicate structures of eye especially retina. Similarly, bioactive lipids are soluble in other lipid solvents such as ethyl acetate, methanol, chloroform, acetone, hexane, isopropanol, methyl-tent-butyl ether (MTBE), or detergent such as Triton X-114. But, all these solvents themselves have potent cytotoxic actions and thus, bioactive lipids dissolved in these solvents showed toxic action on normal cells. In view of this solubility issue and non-availability of a suitable non-toxic solvent to dissolve various bioactive lipids for their appropriate delivery to human tissues, especially to vitreal cavity and retina, no progress has been made in the delivery of various bioactive lipids for various human diseases including DR, AMD, DME and retinopathy of prematurity. Identification and/or development of a suitable solvent for bioactive lipids (since in the absence of a suitable solvent bioactive lipids cannot be injected into human vitreal cavity) is an essential so that their beneficial action can be exploited in an appropriate fashion for the treatment of DR, AMD, DME and retinopathy of prematurity. This is so since, if the solvent used to dissolve bioactive lipids is toxic then it is likely to produce significant side effects especially when these lipids are injected into the human vitreal cavity. Hence, it is critical that a suitable solvent is identified or developed to dissolve bioactive lipids such that the solvent is non-toxic and delivery of bioactive lipids can be performed safely. Furthermore, the solvent used should make bioactive lipids, at least, partially water soluble so that further dilution of the solution is possible to deliver the required amount(s) of the bioactive lipid(s). In the absence of such a suitable water soluble or at least partially water soluble solvent system for dissolving and delivering bioactive lipids, one will not be able to deliver appropriate amounts of bioactive lipid(s) needed to produce the desired actions. In the absence of such a suitable water soluble or at least partially water soluble system, the amount(s) of bioactive lipid(s) delivered to the tissues will be either too high or too low but not appropriate. Thus, development of a suitable solvent system for the delivery of bioactive lipids is critical so that even if accidentally bioactive lipid(s) is injected into normal tissues surrounding vitreal cavity and retina no side effects will occur due to the solvent used for delivering bioactive lipid(s) into the vitreal cavity. This is important since, bioactive lipids by themselves are not toxic to normal cells and to rule out the possibility that the side effects observed are due to the solvent system used for the delivery of bioactive lipids. In this context, the inventor noted after several experiments that dissolving bioactive lipids in pure ethyl alcohol initially followed by subsequent dilutions in sterile saline or PBS (phosphate buffered saline, pH 7.4) such that the final concentration of ethyl alcohol is no more than 0.01% to 0.0001%.

Finally without being bound to any particular theory, it is believed that there is an interaction between the bioactive lipids and the solvent system used to dissolve it which may account for the effectiveness of the treatment. Thus, the solvent system used in some embodiments, which comprises pure ethyl alcohol, and the stabilizing agent are believed to synergistically interact with the bioactive lipids to produce a therapeutic effect which is unexpectedly different than the effect of either bioactive lipid(s) or the solvent agent/stabilizing agent alone.

There are several advantages of bioactive lipid(s) (lipoxins, resolvins, protectins and maresins) treatment according to embodiments disclosed herein. As shown below, a single injection per day once in 4 to 6 weeks for several months (ranging from 6 months to 5 years) at separate times is adequate to produce substantially significant to almost permanent regression of the DR, AMD, DME and retinopathy of prematurity, suppression of the pathological angiogenesis, prevent angiogenesis/neoangiogenesis (formation of new blood vessels) with no or very little recurrence of the pathological angiogenesis. Bioactive lipids and their salts are non-antigenic, are known to be relatively safe in the dosages employed and are stable as prepared and used.

Some embodiments provide a method for treating, preventing, or reversing diabetic retinopathy, age-related macular degeneration, retinopathy of prematurity in children, or diabetic macular edema in a mammal in need thereof, the method comprising administering to the mammal a therapeutically effective amount of a composition according to embodiments described herein.

One embodiment provides methods of inhibiting the growth of new blood vessels that form the basis of pathological angiogenesis leading to DR, AMD, DME and retinopathy of prematurity (a process called as angiogenesis or neoangiogenesis as defined previously above).

Another embodiment provides methods for treating DR, AMD, DME and retinopathy of prematurity and for facilitating visualization of remission of DR, AMD, DME and retinopathy of prematurity which is responsive to treatment, comprising the steps of (a) identifying DR, AMD, DME and retinopathy of prematurity by employing: a fluorescent angiogram; (ii) direct and indirect optic fundal examination of the eye; (iii) optical coherence tomography (OCT) exam that provides cross-sectional images of the retina that shows the thickness of the retina, which will help determine whether fluid has leaked into retinal tissue; (iv) specifically measuring central retinal thickness (CRT), and (v) best-corrected visual acuity (BCVA) measurement; (b) obtaining an initial assessment of DR, AMD, DME and retinopathy of prematurity; (c) injecting into the vitreal cavity of the eye a preparation of bioactive lipid(s) that may comprise a mixture of (i) lipoxins/resolvins/protectins and maresins in a defined proportion and concentration of each of these bioactive lipids solution dissolved in the most suitable solvent and a stabilizing agent; (ii) a solution of at least one bioactive lipid chosen from the group consisting of lipoxin A4, lipoxin B4, resolvin D1, resolvin D2, resolvin E1, resolvin E2, protectin D1, protectin D2, and maresin and (iii) obtaining second and optionally, subsequent status of the DR, AMD, DME, and retinopathy of prematurity by employing: a fluorescent angiogram; direct and indirect optic fundal examination of the eye; optical coherence tomography (OCT) exam that provides cross-sectional images of the retina that shows the thickness of the retina, which will help determine whether fluid has leaked into retinal tissue; measuring central retinal thickness (CRT), and best-corrected visual acuity (BCVA) measurement after predetermined lapses of time; and comparing the initial status of DR, AMD, DME and retinopathy of prematurity with the second and/or subsequent assessments to know the extent of remission of DR, AMD, DME, and retinopathy of prematurity.

Accordingly, certain embodiments provide a method according to the foregoing embodiments, wherein the method further comprises identifying and monitoring remission of diabetic retinopathy, age-related macular degeneration, retinopathy of prematurity in children, or diabetic macular edema using at least one of the following:

i) fluorescent angiogram;

ii) direct or indirect optical fundal examination of the eye;

iii) optical coherence tomography;

iv) central retinal thickness measurement; or

v) best-corrected visual acuity measurement.

In yet another aspect, an embodiment provides methods of treating pathological retinopathy disorders using a solution of a bioactive lipid, or a combination of bioactive lipids, administered intra-vitreally. The methods are as described above with respect to a pathological angiogenesis/retinopathy that is specifically known to occur in DR, AMD, DME, and retinopathy of prematurity. In some of the foregoing embodiments, the administration comprises an intra-vitreal injection. In related embodiments, the administration comprises intra-vitreal delivery by a biodegradable wafer or membrane.

In each of the foregoing embodiments, the bioactive lipid, such as lipoxin A4, lipoxin B4, resolvins, protectins and maresins, is preferably in the form of a free acid or any other suitable salt form, and is preferably administered in combination with a stabilizing agent and the final ethyl alcohol concentration is no more than 0.01 to 0.0001%. The maintenance of the appropriate concentration of ethyl alcohol and the stabilizing agent (e.g., not more than 0.01 to 0.0001%) is believed to contribute to the fact that the bioactive lipids are stable and the solvent and/or the stabilizing agent do not interfere with the anti-angiogenic, anti-inflammatory and cytoprotective actions of the said bioactive lipid. The inventor also observed to his surprise that when the ethyl alcohol concentration is more than 0.01% and less than 0.0001% in any solution wherein bioactive lipid(s) is present, the action of bioactive lipid(s) is suboptimal and became unstable and its (bioactive lipids) anti-angiogenic, anti-inflammatory and cytoprotective actions are not optimal. This is an unexpected observation since, it is never expected that any solvent that is used for dissolving an active chemical could be so critical for the stability and activity of the active chemical that has been dissolved in a given solvent. But, in the case of bioactive lipids this was found to be true. Any increase in the solvent content above 0.01% and lower than 0.0001% in a given solution in which bioactive lipids is present, the activity, stability and its anti-DR, anti-AMD, anti-DME and anti-retinopathy of prematurity actions were altered such that bioactive lipids became relatively inactive, unstable and their beneficial action inefficient. In some embodiments, the amount of the bioactive lipid administered ranges from 1 ng to 100 mg in a volume ranging from 10 μL to 1000 μL. For example, in some embodiments, the amount of the bioactive lipid administered ranges from 5 ng to 75 mg, from 10 ng to 50 mg, from 20 ng to 40 mg, from 100 ng to 1 mg, from 500 ng to 1 mg, or from 750 ng to 1 mg. In some of those embodiments the bioactive lipid is administered in a volume ranging from 20 μL to 750 μL, 30 μL to 600 μL, 50 μL to 500 μL, or from 100 μL to 250 μL.

Thus, as already discussed above, a surprising and novel observation of the inventor is the fact that any increase in the solvent content above 0.01% and lower than 0.0001% in a given solution in which bioactive lipid(s) is present, the activity, stability and its (their) anti-angiogenic, antiinflammatory and cytoprotective actions were altered such that bioactive lipid became relatively inactive, unstable and its (their) anti-DR, anti-AMD, anti-DME and anti-retinopathy of prematurity action(s) inefficient. This surprising observation that the concentration of ethyl alcohol in the final solution containing bioactive lipid(s) and the type of bioactive lipid (such as free acid form) are the critical factors that need specific and particular attention both for stability of the active compound (LXA4 in this instance) and to obtain its beneficial action. These properties of the bioactive lipid free acid is seen only when the solvent ethyl alcohol content in the final solution is between 0.01% to 0.0001% that cannot be anticipated from the prior art. Based on the prior art, those skilled in the art would have anticipated that the injection of bioactive lipid(s) into the vitreal cavity of the eye would result in (i) injury; (ii) inflammation; and (iii) uveitis and/or uveoretinitis (inflammation of uveal structures and retina) due to the formation of toxic metabolites such as lipid peroxides that are known to be toxic to cells; (iii) cause occlusion of blood vessels in the retina and that would have led to the onset of damage to retina, retinal detachment and loss of vision and/or (iv) other deleterious actions. It may also be noted that neuronal cells of the retina are more amenable to lipid peroxidation due to their high content of unsaturated fatty acids. In contrast to these anticipated actions based on the prior art, the inventor noted that bioactive lipid(s) such as lipoxins, resolvins, protectins and maresins when prepared as defined above and in the solvent system as described and administered as outlined, it produced a dramatic beneficial action that is totally unanticipated and against all prediction and produced inhibition, regression and even reversal of only abnormal angiogenesis with no action on normal retinal cells, vascular endothelial cells of retinal vessels, retinal pigment epithelial cells and retinal capillary pericytes and other normal cells of the eye and regression of DR, AMD, DME, and retinopathy of prematurity. This differential action of bioactive lipids only on abnormal or pathological retinopathy but not on normal cells including normal neuronal cells is rather reassuring and unanticipated.

Although certain embodiments are described primarily as it relates to humans, it is envisaged that the methods of certain embodiments are equally applicable to other mammals, including large domesticated mammals (e.g., race horses, breeding cattle) and other smaller domesticated animals (e.g., house pets, dogs).

One embodiment employs bioactive lipids, preferably in the form of free acid. Preferred bioactive lipids include, but are not limited to lipoxins, resolvins, protectins and maresins. Other preferred bioactive lipids include derivatives of the aforementioned bioactive lipids, including glycerides, esters, amides, or phospholipids, or alkylated, alkoxylated, halogenated, sulfonated, or phosphorylated forms. In most preferred embodiments, the bioactive lipid is lipoxin A4, resolvin D1, resolvin E1, protectin D1, maresin 1.

The bioactive lipid is preferably administered in the form of a free acid solution. Other suitable forms include in the form of a salt solution. Suitable salt include salts of a bioactive lipid with cation of a small organic group (ammonium) or a small inorganic group (e.g., an alkali metal or alkali earth metal). Preferred referred salts are those between a bioactive lipid and an alkali metal (e.g., lithium, sodium, potassium), an alkali earth metal (e.g., magnesium, calcium) or a multivalent metal (e.g., manganese, iron, copper, aluminium, zinc, chromium, cobalt, nickel). Most preferred are free acids of the said bioactive lipid. Combinations of free acids or salts may also be employed. When the bioactive lipids or bioactive lipid free acids are administered in a suitable solvent as discussed above, the solution may be formed into an emulsion.

In one aspect, one embodiment provides pharmaceutical compositions comprising a bioactive lipid, or a bioactive lipid salt, a bioactive lipid acid, and anti-VEGF antibody and corticosteroid in a solution, or in an emulsion. In certain embodiments, the composition is a solution or emulsion. The bioactive lipid and anti-VEGF antibody and corticosteroid (for example triamcinolone) may be separate chemical moieties combined in a solution or emulsion, or they may be covalently conjugated. In certain specific embodiments, the corticosteroid is triamcinolone. The anti-VEGF antibody may be mixed with the bioactive lipid solution described above, either to form a new solution or to form an emulsion, or they may be chemically conjugated to the bioactive lipid of an embodiment via standard chemistries. Thus, the term “derivative” as used herein as it relates to bioactive lipids includes such conjugates. Preferably the bioactive lipid solution is mixed with such an anti-VEGF antibody in a ratio of at least about 2:1, or about 1:1 or about 1:1.5, or about 1:2 or about 1:3 (volume/volume or Mol:Mol). Thus, in some embodiments, the bioactive lipid and anti-VEGF antibody are present in a molar ratio of about 2:1, about 1:1, about 1:1.5, about 1:2, or about 1:3. In some embodiments the bioactive lipid and anti-VEGF antibody are present in a molar ratio ranging from about 2:1 to about 1:3. Most preferably the ratio is between 1:1.5 and 1:3 (volume/volume). The bioactive lipid solution may be safely administered to a typical patient with DR, AMD, DME, retinopathy of prematurity in an amount of about 1 μg to 50 mg in a volume of about 10 μl to 1000 μl or more, but the attending physician should consider all relevant medical factors in determining the appropriate dosage for any specific patient. The preferred bioactive lipid(s) solution and ratios of such a product (a combination of a bioactive lipid and anti-VEGF antibody) are as disclosed above. Preferably the final concentration of the bioactive lipid(s) in such a product is at least 5%, preferably at least 25%, and most preferably about 25-75% w/w, v/v or v/w. Accordingly, in some of the foregoing embodiments, the concentration of bioactive lipid is at least 5%, at least 15%, or at least 25% w/w, v/v or v/w. In some embodiments, the concentration of bioactive lipid ranges from about 25% to 75%, about 30% to 60%, or about 40% to 50% w/w.

In some of the foregoing embodiments, the composition further comprises an anti-VEGF antibody, a corticosteroid, or combinations thereof In another aspect, an embodiment provides pharmaceutical compositions comprising a bioactive lipid(s), or a bioactive lipid(s) salt, and anti-VEGF antibody and/or corticosteroid an in solution, or in an emulsion. The bioactive lipid and anti-VEGF antibody and/or corticosteroid may be separate chemical moieties combined in the solution or emulsion, or they may be covalently conjugated. In certain embodiments, the anti-VEGF antibody, the corticosteroid, or both are covalently conjugated to the bioactive lipid (i.e., a bioactive lipid derivative). The preferred bioactive lipid(s) solution and ratios of such a product (a combination of a bioactive lipid(s) and anti-VEGF antibody and/or corticosteroid) is at least 5%, preferably at least 25%, and most preferably about 25-75%.

In another aspect, an embodiment provides pharmaceutical compositions comprising a bioactive lipid, or a bioactive lipid salt; anti-VEGF antibody and/or corticosteroid drug may be separate chemical moieties combined in a solution or emulsion, or they may be covalently conjugated. The anti-VEGF antibody and/or corticosteroid drug may be mixed with the bioactive lipid(s) solution described above, either to form a new solution or to form an emulsion, or they may be chemically conjugated to the bioactive lipid(s) of the invention via standard chemistries. Preferably the bioactive lipid(s) solution is mixed with such an anti-VEGF antibody and/or corticosteroid drug in a ratio of at least about 1:1:1, or about 10:1:1, or about 1:10:1, or about 1:1:10 or in any combination or ratio (volume/volume/volume or Mol:Mol:Mol). Thus, in some of the foregoing embodiments, the bioactive lipid, anti-VEGF antibody, and corticosteroid are present in a molar or volumetric ratio of at least 1:1:1, about 10:1:1, about 1:10:1 or about 1:1:10. In some embodiments, the bioactive lipid, anti-VEGF antibody, and corticosteroid are present in a molar or volumetric ratio ranging from about 1:1:1 to about 10:1:1 to about 1:10:1 to about 1:1:10. The bioactive lipid(s) solution may be safely administered to a typical patient with DR, AMD, DME, retinopathy of prematurity in an amount of about 1 μg to 50 mg in a volume of about 10 μl to 1000 μl or more as the case may be, but the attending physician should consider all relevant medical factors in determining the appropriate dosage for any specific patient. The preferred bioactive lipid(s) solution and ratios of such a product (a combination of a bioactive lipid(s) and anti-VEGF/corticosteroid drug) are as disclosed above. Preferably the final concentration of the bioactive lipid(s) in such a product is at least 5%, preferably at least 25%, and most preferably about 25-75%.

The bioactive lipid(s) solutions of certain embodiments are preferably administered intra-vitreally. In the case of a DR, the bioactive lipid(s) solutions are injected into the vitreal cavity of the eye before or after photocoagulation or before or after any suitable surgery needed for retinopathy. It is also envisaged that in the treatment of DR, AMD, DME and retinopathy of prematurity, bioactive lipid(s) solution is delivered into the vitreal cavity y incorporating bioactive lipid(s) in a biodegradable wafer or membranes for slowly, steady and predictable amounts of bioactive lipid(s) to be delivered into the vitreal cavity anywhere from 24 hours to 1 week or even up to 10 weeks. The delivery of bioactive lipid(s) is delivered into the vitreal cavity of eye by inserting the biodegradable membrane/wafer containing bioactive lipid(s) at the time of any surgery to the eye.

Thus, in another aspect, one embodiment provides pharmaceutical compositions comprising a bioactive lipid(s) (including lipoxins, resolvins, protectins and maresins-each compound alone or in combination of two or three or more), a bioactive lipid(s) salt, and a pharmaceutical agent known in the art for the treatment of DR, AMD, DME and retinopathy of prematurity, either in solution, or in an emulsion. The bioactive lipid(s) and other pharmaceutical agent may be separate chemical moieties combined in the solution or emulsion, or they may be covalently conjugated. The preferred pharmaceutical agents as disclosed above. Preferably the final concentration of the bioactive lipid in such a product is at least 5%, preferably at least 15%, and most preferably at least 25%. The product may contain substantially more bioactive lipid(s), up to 100% without any significant side effects.

Suitable routes of administration include, but are not limited to, oral, intravenous, rectal, aerosol, parenteral, ophthalmic, pulmonary, transmucosal, transdermal, vaginal, otic, nasal, and topical administration. In addition, by way of example only, parenteral delivery includes intramuscular, subcutaneous, intravenous, intramedullary injections, as well as intrathecal, direct intraventricular, intraperitoneal, intralymphatic, and intranasal injections.

In certain embodiments, a composition as described herein is administered in a local rather than systemic manner, for example, via injection of the composition F directly into an organ, often in a depot preparation or sustained release formulation. In specific embodiments, long acting formulations are administered by implantation (for example subcutaneously or intramuscularly) or by intramuscular injection. Furthermore, in other embodiments, the drug is delivered in a targeted drug delivery system, for example, in a liposome coated with organ-specific antibody. In such embodiments, the liposomes are targeted to and taken up selectively by the organ. In yet other embodiments, the composition as described herein is provided in the form of a rapid release formulation, in the form of an extended release formulation, or in the form of an intermediate release formulation. In yet other embodiments, the composition described herein is administered topically.

The compositions according to the invention are effective over a wide dosage range. For example, in the treatment of adult humans, dosages from 0.01 to 1000 mg, from 0.5 to 100 mg, from 1 to 50 mg per day, and from 5 to 40 mg per day are examples of dosages that are used in some embodiments. An exemplary dosage is 10 to 30 mg per day. The exact dosage will depend upon the route of administration, the form in which the composition is administered, the subject to be treated, the body weight of the subject to be treated, and the preference and experience of the attending physician.

In some embodiments, a composition of the invention is administered in a single dose. Typically, such administration will be by injection, e.g., intravenous injection, in order to introduce the agent quickly. However, other routes are used as appropriate. A single dose of a composition of the invention may also be used for treatment of an acute condition.

In some embodiments, a composition of the invention is administered in multiple doses. In some embodiments, dosing is about once, twice, three times, four times, five times, six times, or more than six times per day. In other embodiments, dosing is about once a month, once every two weeks, once a week, or once every other day. In another embodiment a composition of the invention and another agent are administered together about once per day to about 6 times per day. In another embodiment the administration of a composition of the invention and an agent continues for less than about 7 days. In yet another embodiment the administration continues for more than about 6, 10, 14, 28 days, two months, six months, or one year. In some cases, continuous dosing is achieved and maintained as long as necessary.

Administration of the compositions of the invention may continue as long as necessary. In some embodiments, a composition of the invention is administered for more than 1, 2, 3, 4, 5, 6, 7, 14, or 28 days. In some embodiments, a composition of the invention is administered for less than 28, 14, 7, 6, 5, 4, 3, 2, or 1 day. In some embodiments, a composition of the invention is administered chronically on an ongoing basis, e.g., for the treatment of chronic effects.

In some embodiments, the compositions of the invention are administered in dosages. It is known in the art that due to intersubject variability in pharmacokinetics, individualization of dosing regimen is necessary for optimal therapy. Dosing for a composition of the invention may be found by routine experimentation in light of the instant disclosure.

In some embodiments, the compositions described herein are formulated into pharmaceutical compositions. In specific embodiments, pharmaceutical compositions are formulated in a conventional manner using one or more physiologically acceptable carriers comprising excipients and auxiliaries which facilitate processing of the active compositions into preparations which can be used pharmaceutically. Proper formulation is dependent upon the route of administration chosen. Any pharmaceutically acceptable techniques, carriers, and excipients are used as suitable to formulate the pharmaceutical compositions described herein: Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999).

Provided herein are pharmaceutical compositions comprising a pharmaceutically acceptable diluent(s), excipient(s), or carrier(s). In certain embodiments, the compositions described are administered as pharmaceutical compositions mixed with other active ingredients, as in combination therapy. Encompassed herein are all combinations of actives set forth in the combination therapies section below and throughout this disclosure.

A pharmaceutical composition, as used herein, refers to a mixture of a composition according to any of the embodiments disclosed herein with other chemical components, such as carriers, stabilizers, diluents, dispersing agents, suspending agents, thickening agents, and/or excipients. In certain embodiments, the pharmaceutical composition facilitates administration of the composition to an organism. In some embodiments, practicing the methods of treatment or use provided herein, therapeutically effective amounts of the composition provided herein are administered in a pharmaceutical composition to a mammal having a disease, disorder or medical condition to be treated. In specific embodiments, the mammal is a human. In certain embodiments, therapeutically effective amounts vary depending on the severity of the disease, the age and relative health of the subject, the potency of the composition used and other factors. The compositions described herein are used singly or in combination with one or more therapeutic agents as components of mixtures.

In one embodiment, a composition is formulated in an aqueous solution. In specific embodiments, the aqueous solution is selected from, by way of example only, a physiologically compatible buffer, such as Hank's solution, Ringer's solution, or physiological saline buffer. In other embodiments, a composition is formulated for transmucosal administration. In specific embodiments, transmucosal formulations include penetrants that are appropriate to the barrier to be permeated. In still other embodiments wherein the compounds described herein are formulated for other parenteral injections, appropriate formulations include aqueous or non-aqueous solutions. In specific embodiments, such solutions include physiologically compatible buffers and/or excipients.

In another embodiment, compounds described herein are formulated for oral administration. Compounds described herein are formulated by combining the active compounds with, e.g., pharmaceutically acceptable carriers or excipients. In various embodiments, the compounds described herein are formulated in oral dosage forms that include, by way of example only, tablets, powders, pills, dragees, capsules, liquids, gels, syrups, elixirs, slurries, suspensions and the like.

In certain embodiments, pharmaceutical preparations for oral use are obtained by mixing one or more solid excipient with one or more of the compounds described herein, optionally grinding the resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as: for example, maize starch, wheat starch, rice starch, potato starch, gelatin, gum tragacanth, methylcellulose, microcrystalline cellulose, hydroxypropylmethylcellulose, sodium carboxymethylcellulose; or others such as: polyvinylpyrrolidone (PVP or povidone) or calcium phosphate. In specific embodiments, disintegrating agents are optionally added. Disintegrating agents include, by way of example only, cross-linked croscarmellose sodium, polyvinylpyrrolidone, agar, or alginic acid or a salt thereof such as sodium alginate.

In one embodiment, dosage forms, such as dragee cores and tablets, are provided with one or more suitable coating. In specific embodiments, concentrated sugar solutions are used for coating the dosage form. The sugar solution, optionally contain additional components, such as by way of example only, gum arabic, talc, polyvinylpyrrolidone, carbopol gel, polyethylene glycol, and/or titanium dioxide, lacquer solutions, and suitable organic solvents or solvent mixtures. Dyestuffs and/or pigments are also optionally added to the coatings for identification purposes. Additionally, the dyestuffs and/or pigments are optionally utilized to characterize different combinations of active compound doses.

In certain embodiments, therapeutically effective amounts of at least one of the compounds described herein are formulated into other oral dosage forms. Oral dosage forms include push-fit capsules made of gelatin, as well as soft, sealed capsules made of gelatin and a plasticizer, such as glycerol or sorbitol. In specific embodiments, push-fit capsules contain the active ingredients in admixture with one or more filler. Fillers include, by way of example only, lactose, binders such as starches, and/or lubricants such as talc or magnesium stearate and, optionally, stabilizers. In other embodiments, soft capsules, contain one or more active compound that is dissolved or suspended in a suitable liquid. Suitable liquids include, by way of example only, one or more fatty oil, liquid paraffin, or liquid polyethylene glycol. In addition, stabilizers are optionally added.

In other embodiments, therapeutically effective amounts of at least one of the compounds described herein are formulated for buccal or sublingual administration. Formulations suitable for buccal or sublingual administration include, by way of example only, tablets, lozenges, or gels. In still other embodiments, the compounds described herein are formulated for parental injection, including formulations suitable for bolus injection or continuous infusion. In specific embodiments, formulations for injection are presented in unit dosage form (e.g., in ampoules) or in multi-dose containers. Preservatives are, optionally, added to the injection formulations. In still other embodiments, the pharmaceutical compositions are formulated in a form suitable for parenteral injection as sterile suspensions, solutions or emulsions in oily or aqueous vehicles. Parenteral injection formulations optionally contain formulatory agents such as suspending, stabilizing and/or dispersing agents. In specific embodiments, pharmaceutical formulations for parenteral administration include aqueous solutions of the active compositions in water-soluble form. In additional embodiments, suspensions of the active composition are prepared as appropriate oily injection suspensions. Suitable lipophilic solvents or vehicles for use in the pharmaceutical compositions described herein include, by way of example only, fatty oils such as sesame oil, or synthetic fatty acid esters, such as ethyl oleate or triglycerides, or liposomes. In certain specific embodiments, aqueous injection suspensions contain substances which increase the viscosity of the suspension, such as sodium carboxymethyl cellulose, sorbitol, or dextran. Optionally, the suspension contains suitable stabilizers or agents which increase the solubility of the compositions to allow for the preparation of highly concentrated solutions. Alternatively, in other embodiments, the active ingredient is in powder form for constitution with a suitable vehicle, e.g., sterile pyrogen-free water, before use.

In still other embodiments, the compositions are administered topically. The compositions described herein are formulated into a variety of topically administrable forms, such as solutions, suspensions, lotions, gels, pastes, medicated sticks, balms, creams or ointments. Such pharmaceutical compositions optionally contain solubilizers, stabilizers, tonicity enhancing agents, buffers and preservatives.

In yet other embodiments, the compositions are formulated for transdermal administration. In specific embodiments, transdermal formulations employ transdermal delivery devices and transdermal delivery patches and can be lipophilic emulsions or buffered, aqueous solutions, dissolved and/or dispersed in a polymer or an adhesive. In various embodiments, such patches are constructed for continuous, pulsatile, or on demand delivery of pharmaceutical agents. In additional embodiments, the transdermal delivery of the compositions is accomplished by means of iontophoretic patches and the like. In certain embodiments, transdermal patches provide controlled delivery of the compositions. In specific embodiments, the rate of absorption is slowed by using rate-controlling membranes or by trapping the composition within a polymer matrix or gel. In alternative embodiments, absorption enhancers are used to increase absorption. Absorption enhancers or carriers include absorbable pharmaceutically acceptable solvents that assist passage through the skin. For example, in one embodiment, transdermal devices are in the form of a bandage comprising a backing member, a reservoir containing the compostion optionally with carriers, optionally a rate controlling barrier to deliver the composition to the skin of the host at a controlled and predetermined rate over a prolonged period of time, and means to secure the device to the skin.

In other embodiments, the compositions are formulated for administration by inhalation. Various forms suitable for administration by inhalation include, but are not limited to, aerosols, mists or powders. Some embodiments of compositions as disclosed herein are conveniently delivered in the form of an aerosol spray presentation from pressurized packs or a nebulizer, with the use of a suitable propellant (e.g., dichlorodifluoromethane, trichlorofluoromethane, dichlorotetrafluoroethane, carbon dioxide or other suitable gas). In specific embodiments, the dosage unit of a pressurized aerosol is determined by providing a valve to deliver a metered amount. In certain embodiments, capsules and cartridges of, such as, by way of example only, gelatin for use in an inhaler or insufflator is formulated containing a powder mix of the compound and a suitable powder base such as lactose or starch.

In still other embodiments, the compositions are formulated in rectal compositions such as enemas, rectal gels, rectal foams, rectal aerosols, suppositories, jelly suppositories, or retention enemas, containing conventional suppository bases such as cocoa butter or other glycerides, as well as synthetic polymers such as polyvinylpyrrolidone, PEG, and the like. In suppository forms of the compositions, a low-melting wax such as, but not limited to, a mixture of fatty acid glycerides, optionally in combination with cocoa butter is first melted.

In certain embodiments, pharmaceutical compositions are formulated in any conventional manner using one or more physiologically acceptable carriers comprising excipients and auxiliaries which facilitate processing of the active compounds into preparations which can be used pharmaceutically. Proper formulation is dependent upon the route of administration chosen. Any pharmaceutically acceptable techniques, carriers, and excipients are optionally used as suitable. Pharmaceutical compositions are manufactured in a conventional manner, such as, by way of example only, by means of conventional mixing, dissolving, granulating, dragee-making, levigating, emulsifying, encapsulating, entrapping or compression processes.

The bioactive lipid may be in free-acid or free-base form, or in a pharmaceutically acceptable salt form. In addition, the methods and pharmaceutical compositions described herein include the use of N-oxides, crystalline forms (also known as polymorphs), as well as active metabolites of these compounds having the same type of activity. All tautomers of the compounds described herein are included within the scope of the bioactive lipids presented herein. Additionally, the bioactive lipids described herein encompass unsolvated as well as solvated forms with pharmaceutically acceptable solvents such as water, ethanol, and the like. The solvated forms of the compounds presented herein are also considered to be disclosed herein. In addition, the compositions optionally include other medicinal or pharmaceutical agents, carriers, adjuvants, such as preserving, stabilizing, wetting or emulsifying agents, solution promoters, salts for regulating the osmotic pressure, buffers, and/or other therapeutically valuable substances.

Methods for the preparation of compositions comprising the compounds described herein include formulating the compounds with one or more inert, pharmaceutically acceptable excipients or carriers to form a solid, semi-solid or liquid. Solid compositions include, but are not limited to, powders, tablets, dispersible granules, capsules, cachets, and suppositories. Liquid compositions include solutions in which a compound is dissolved, emulsions comprising a compound, or a solution containing liposomes, micelles, or nanoparticles comprising a compound as disclosed herein. Semi-solid compositions include, but are not limited to, gels, suspensions and creams. The form of the pharmaceutical compositions described herein include liquid solutions or suspensions, solid forms suitable for solution or suspension in a liquid prior to use, or as emulsions. These compositions also optionally contain minor amounts of nontoxic, auxiliary substances, such as wetting or emulsifying agents, pH buffering agents, and so forth.

In some embodiments, a composition illustratively takes the form of a liquid where the bioactive lipids are present in solution, in suspension or both. In some embodiments, a liquid composition includes a gel formulation. In other embodiments, the liquid composition is aqueous.

In certain embodiments, useful aqueous suspensions contain one or more polymers as suspending agents. Useful polymers include water-soluble polymers such as cellulosic polymers, e.g., hydroxypropyl methylcellulose, and water-insoluble polymers such as cross-linked carboxyl-containing polymers. Certain pharmaceutical compositions described herein comprise a mucoadhesive polymer, selected for example from carboxymethylcellulose, carbomer (acrylic acid polymer), poly(methylmethacrylate), polyacrylamide, polycarbophil, acrylic acid/butyl acrylate copolymer, sodium alginate and dextran.

Useful pharmaceutical compositions also, optionally, include solubilizing agents to aid in solubility components of the composition. The term “solubilizing agent” generally includes agents that result in formation of a micellar solution or a true solution of the agent. Certain acceptable nonionic surfactants, for example polysorbate 80, are useful as solubilizing agents, as can ophthalmically acceptable glycols, polyglycols, e.g., polyethylene glycol 400, and glycol ethers.

Furthermore, useful pharmaceutical compositions optionally include one or more pH adjusting agents or buffering agents, including acids such as acetic, boric, citric, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the composition in an acceptable range.

Additionally, useful compositions also, optionally, include one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate.

Other useful pharmaceutical compositions optionally include one or more preservatives to inhibit microbial activity. Suitable preservatives include mercury-containing substances such as merfen and thiomersal; stabilized chlorine dioxide; and quaternary ammonium compounds such as benzalkonium chloride, cetyltrimethylammonium bromide and cetylpyridinium chloride.

Still other useful compositions include one or more surfactants to enhance physical stability or for other purposes. Suitable nonionic surfactants include polyoxyethylene fatty acid glycerides and vegetable oils, e.g., polyoxyethylene (60) hydrogenated castor oil; and polyoxyethylene alkylethers and alkylphenyl ethers, e.g., octoxynol 10, octoxynol 40.

Still other useful compositions include one or more antioxidants to enhance chemical stability where required. Suitable antioxidants include, by way of example only, ascorbic acid and sodium metabisulfite.

In certain embodiments, aqueous suspension compositions are packaged in single-dose non-reclosable containers. Alternatively, multiple-dose reclosable containers are used, in which case it is typical to include a preservative in the composition.

In alternative embodiments, other delivery systems for hydrophobic compositions are employed. Liposomes and emulsions are examples of delivery vehicles or carriers useful herein. In certain embodiments, organic solvents such as N-methylpyrrolidone are also employed. In additional embodiments, the compounds described herein are delivered using a sustained-release system, such as semipermeable matrices of solid hydrophobic polymers containing the therapeutic agent. Various sustained-release materials are useful herein. In some embodiments, sustained-release capsules release the components of the composition for a few weeks up to over 100 days. Depending on the chemical nature and the biological stability of the therapeutic reagent, additional strategies for protein stabilization are employed.

In certain embodiments, the formulations described herein comprise one or more antioxidants, metal chelating agents, thiol containing compounds and/or other general stabilizing agents. Examples of such stabilizing agents, include, but are not limited to: (a) about 0.5% to about 2% w/v glycerol, (b) about 0.1% to about 1% w/v methionine, (c) about 0.1% to about 2% w/v monothioglycerol, (d) about 1 mM to about 10 mM EDTA, (e) about 0.01% to about 2% w/v ascorbic acid, (f) 0.003% to about 0.02% w/v polysorbate 80, (g) 0.001% to about 0.05% w/v. polysorbate 20, (h) arginine, (i) heparin, (j) dextran sulfate, (k) cyclodextrins, (1) pentosan polysulfate and other heparinoids, (m) divalent cations such as magnesium and zinc; or (n) combinations thereof.

In some embodiments, the concentration of the one or more bioactive lipid is less than 100%, 90%, 80%, 70%, 60%, 50%, 40%, 30%, 20%, 19%, 18%, 17%, 16%, 15%, 14%, 13%, 12%, 11%, 10%, 9%, 8%, 7%, 6%, 5%, 4%, 3%, 2%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.0001% w/w, w/v or v/v.

In some embodiments, the concentration of the one or more bioactive lipid is greater than 90%, 80%, 70%, 60%, 50%, 40%, 30%, 20%, 19.75%, 19.50%, 19.25%, 19%, 18.75%, 18.50%, 18.25%, 18%, 17.75%, 17.50%, 17.25%, 17%, 16.75%, 16.50%, 16.25%, 16%, 15.75%, 15.50%, 15.25%, 15%, 14.75%, 14.50%, 14.25%, 14%, 13.75%, 13.50%, 13.25%, 13%, 12.75%, 12.50%, 12.25%, 12%, 11.75%, 11.50%, 11.25%, 11%, 10.75%, 10.50%, 10.25%, 10%, 9.75%, 9.50%, 9.25%, 9%, 8.75%, 8.50%, 8.25%, 8%, 7.75%, 7.50%, 7.25%, 7%, 6.75%, 6.50%, 6.25%, 6%, 5.75%, 5.50%, 5.25%, 5%, 4.75%, 4.50%, 4.25%, 4%, 3.75%, 3.50%, 3.25%, 3%, 2.75%, 2.50%, 2.25%, 2%, 1.75%, 1.50%, 125% , 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.0001% w/w, w/v, or v/v.

In some embodiments, the concentration of the one or more bioactive lipid is in the range from approximately 0.0001% to approximately 50%, approximately 0.001% to approximately 40%, approximately 0.01% to approximately 30%, approximately 0.02% to approximately 29%, approximately 0.03% to approximately 28%, approximately 0.04% to approximately 27%, approximately 0.05% to approximately 26%, approximately 0.06% to approximately 25%, approximately 0.07% to approximately 24%, approximately 0.08% to approximately 23%, approximately 0.09% to approximately 22%, approximately 0.1% to approximately 21%, approximately 0.2% to approximately 20%, approximately 0.3% to approximately 19%, approximately 0.4% to approximately 18%, approximately 0.5% to approximately 17%, approximately 0.6% to approximately 16%, approximately 0.7% to approximately 15%, approximately 0.8% to approximately 14%, approximately 0.9% to approximately 12%, approximately 1% to approximately 10% w/w, w/v or v/v.

In some embodiments, the concentration of the one or more bioactive lipid is in the range from approximately 0.001% to approximately 10%, approximately 0.01% to approximately 5%, approximately 0.02% to approximately 4.5%, approximately 0.03% to approximately 4%, approximately 0.04% to approximately 3.5%, approximately 0.05% to approximately 3%, approximately 0.06% to approximately 2.5%, approximately 0.07% to approximately 2%, approximately 0.08% to approximately 1.5%, approximately 0.09% to approximately 1%, approximately 0.1% to approximately 0.9% w/w, w/v or v/v.

In some embodiments, the amount of the one or more bioactive lipid is equal to or less than 10 g, 9.5 g, 9.0 g, 8.5 g, 8.0 g, 7.5 g, 7.0 g, 6.5 g, 6.0 g, 5.5 g, 5.0 g, 4.5 g, 4.0 g, 3.5 g, 3.0 g, 2.5 g, 2.0 g, 1.5 g, 1.0 g, 0.95 g, 0.9 g, 0.85 g, 0.8 g, 0.75 g, 0.7 g, 0.65 g, 0.6 g, 0.55 g, 0.5 g, 0.45 g, 0.4 g, 0.35 g, 0.3 g, 0.25 g, 0.2 g, 0.15 g, 0.1 g, 0.09 g, 0.08 g, 0.07 g, 0.06 g, 0.05 g, 0.04 g, 0.03 g, 0.02 g, 0.01 g, 0.009 g, 0.008 g, 0.007 g, 0.006 g, 0.005 g, 0.004 g, 0.003 g, 0.002 g, 0.001 g, 0.0009 g, 0.0008 g, 0.0007 g, 0.0006 g, 0.0005 g, 0.0004 g, 0.0003 g, 0.0002 g, or 0.0001 g.

In some embodiments, the amount of the one or more bioactive lipid is more than 0.0001 g, 0.0002 g, 0.0003 g, 0.0004 g, 0.0005 g, 0.0006 g, 0.0007 g, 0.0008 g, 0.0009 g, 0.001 g, 0.0015 g, 0.002 g, 0.0025 g, 0.003 g, 0.0035 g, 0.004 g, 0.0045 g, 0.005 g, 0.0055 g, 0.006 g, 0.0065 g, 0.007 g, 0.0075 g, 0.008 g, 0.0085 g, 0.009 g, 0.0095 g, 0.01 g, 0.015 g, 0.02 g, 0.025 g, 0.03 g, 0.035 g, 0.04 g, 0.045 g, 0.05 g, 0.055 g, 0.06 g, 0.065 g, 0.07 g, 0.075 g, 0.08 g, 0.085 g, 0.09 g, 0.095 g, 0.1 g, 0.15 g, 0.2 g, 0.25 g, 0.3 g, 0.35 g, 0.4 g, 0.45 g, 0.5 g, 0.55 g, 0.6 g, 0.65 g, 0.7 g, 0.75 g, 0.8 g, 0.85 g, 0.9 g, 0.95 g, 1 g, 1.5 g, 2 g, 2.5, 3 g, 3.5, 4 g, 4.5 g, 5 g, 5.5 g, 6 g, 6.5 g, 7 g, 7.5 g, 8 g, 8.5 g, 9 g, 9.5 g, or 10 g.

In some embodiments, the amount of the one or more bioactive lipid ranges from 0.0001 to 10 g, 0.0005 to 9 g, 0.001 to 8 g, 0.005 to 7 g, 0.01 to 6 g, 0.05 to 5 g, 0.1 to 4 g, 0.5 to 4 g, or 1 to 3 g.

The following examples illustrate some preferred modes of practicing embodiments of the present invention, but are not intended to limit the scope of the claimed embodiments of the invention. Alternative materials and methods may be utilized to obtain similar results.

### EXAMPLES

### Example 1

### Preparation of Lipdxin A4 (Free Acid) Mixture

Pure LXA4 (free acid form) was obtained from Sigma chemicals, USA or Cayman Chemicals, USA and was dissolved in 100% ethyl alcohol. The resultant solution was diluted in normal saline or phosphate buffered saline (PBS), pH 7.4) such that the final concentration of ethyl alcohol ranged from 0.01% to 0.001%. The final concentration of LXA4 in these solutions was approximately 25% to 90%. The LXA4 solution was mixed with a stabilizing agent [in the form of human albumin]. The final concentration of the stabilizing agent ranged from 0.01 to 0.001%. The mixture was prepared under strict sterile conditions.

### Example 2

### Modification of Bioactive Lipid

The bioactive lipid is modified by covalent conjugation (e.g., amide bond) to anti-VEGF antibody, anti-EGF antibody, angiostatin, endostatin or other anti-angiogenic substances (especially when the anti-angiogenic action is not needed to its fullest extent or only partial anti-angiogenic action is needed) in a molar or volumetric ratio of at least about 1:1:1, about 10:1:1, about 1:10:1, about 1:1:10 or in any combination or ratio. The mixtures are prepared under strict sterile conditions prior to use.

### Example 3

### Administration of Pharmaceutical Compositions to Patients

Patients were administered bioactive lipid preparation in the hospital. A complete clinical examination and biochemical assessment of the patient was done including a fluorescent angiogram; direct and indirect optic fundal examination of the eye; optical coherence tomography (OCT) exam that provides cross-sectional images of the retina that shows the thickness of the retina, which will help determine whether fluid has leaked into retinal tissue; measuring central retinal thickness (CRT), and best-corrected visual acuity (BCVA) measurement. Then the bioactive lipid composition was injected into the vitreal cavity (intravitreal injection) once in 4 weeks or weekly. Depending on the response of DR, AMD, DME, retinopathy of prematurity, the number of doses can be repeated (once in a week or once in 4 weeks or once in months as the case may be) until DR, AMD, DME, and retinopathy of prematurity regresses to the satisfaction of the treating physician.

### Example 4

### Assessing DR, AMD, DME and Retinopathy of Prematurity Following Treatment with Bioactive Lipid(s)

The degree of regression of DR, AMD, DME, and retinopathy of prematurity is assessed by performing a fluorescent angiogram; direct and indirect optic fundal examination of the eye; optical coherence tomography (OCT) exam that provides cross-sectional images of the retina that shows the thickness of the retina, which will help determine whether fluid has leaked into retinal tissue; measuring central retinal thickness (CRT), and best-corrected visual acuity (BCVA) measurement after 14 weeks of administration of the bioactive lipid(s) composition and comparing it to the pre-injection examination.

### Example 5

### Study 1: Injection of Bioactive Lipid Free Acid Composition

10 experimental animals with DR, AMD, DME, retinopathy of prematurity (each group consisting of 10 animals: total 40 animals) were treated by direct intravitreal injection of LXA4 free acid form of composition according the examples 1-4 in doses ranging from 10.0 μg/day once in a week for 4 weeks. All 40 of these animals showed significant reduction in the degree of DR, AMD, DME, and retinopathy of prematurity. Control animals that received only carrier (ethanol/albumin/ethanol+albumin did not show any regression of DR, AMD, DME, retinopathy of prematurity. This study was also repeated with different doses of LXA4 using 5 ng, 10 ng, 15 ng, 20 ng, 50 ng, 100 ng, 200 ng, 500 ng, 1 μg, 5 μg, 10 μg, 20.0 μg, 25.0 μg, 50.0 μg, and 100.0 μg per week for 4 weeks; once in 4 weeks for 3 months; and in all these studies and at all the doses tested and for different periods of time a substantial regression of DR, AMD, DME, retinopathy of prematurity was noted.

### Example 6

### Study 2: Injection of Bioactive Lipid Free Acid Composition

In another study, this was similar to the study given in Example 5, except that in this instance different bioactive lipids singly and in combination were tested. Thus, in this study, experimental animals received LXA4, resolvin E1, resolvin D1, protectin D1, maresin 1 or a combination of LXA4+resolvin E1+protectin D1+maresin 1 (in the ratio of 1:1:1:1) were given with appropriate control groups. In this study, different doses of bioactive lipids were used either 5 ng, 10 ng, 15 ng, 20 ng, 50 ng, 100 ng, 200 ng, 500 ng, 1 μg, 5 μg or ranging from 10.0 μg to 100.0 μg per week for 4 weeks; once in 4 weeks for 3 months. In this study also and at all the doses tested and for different periods of time a substantial regression of DR, AMD, DME, retinopathy of prematurity was noted.

### Example 7

### Study 3: Injection of Bioactive Lipid Free Acid Composition

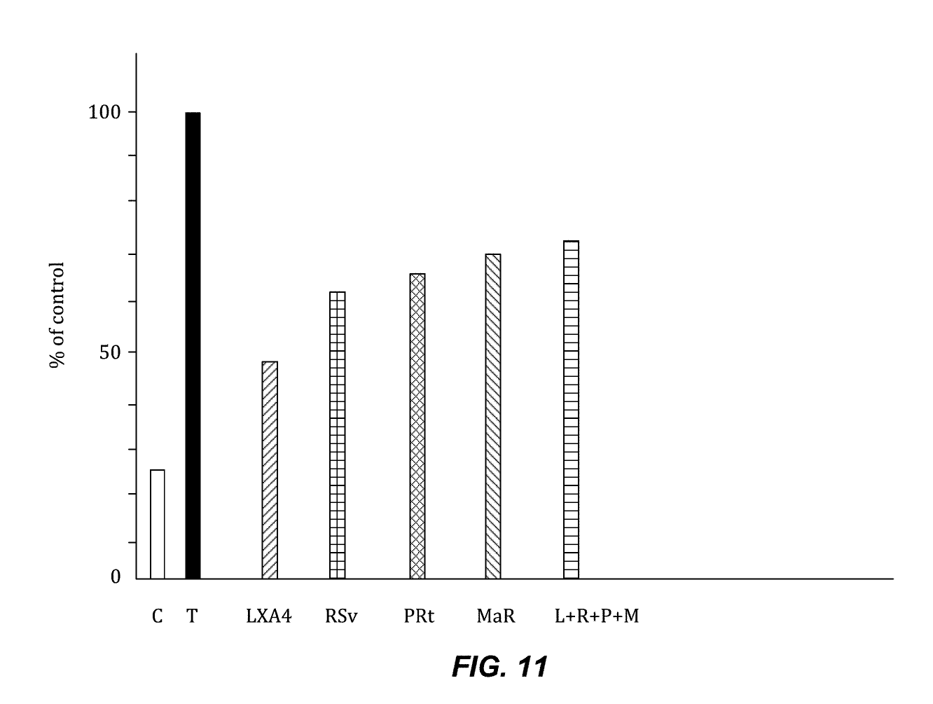

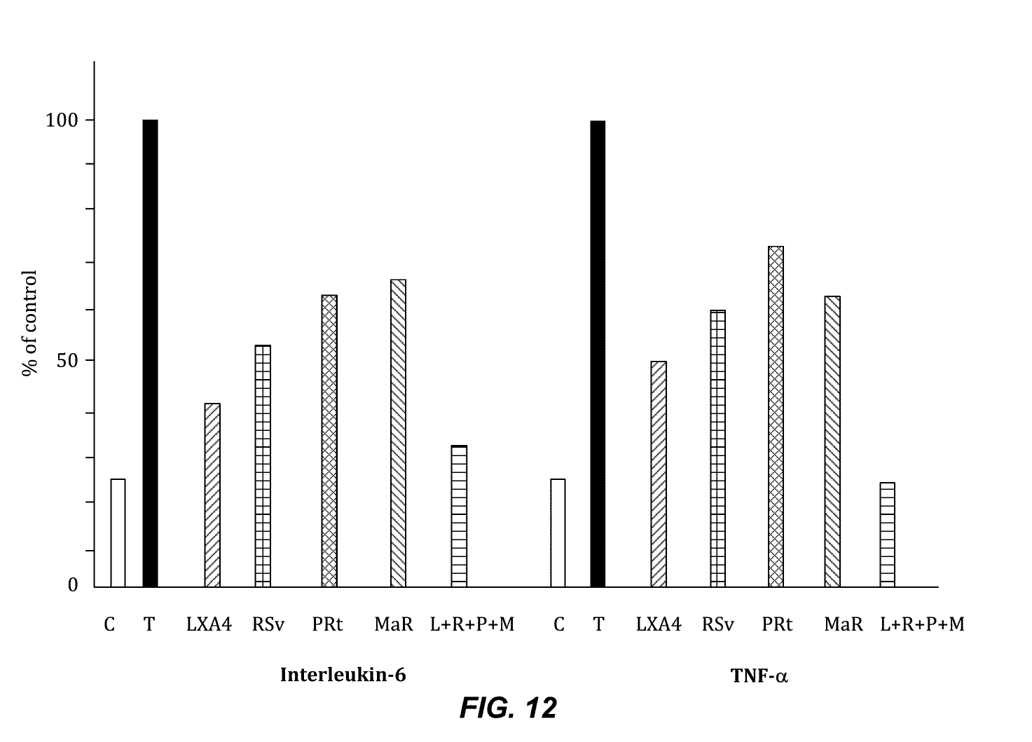

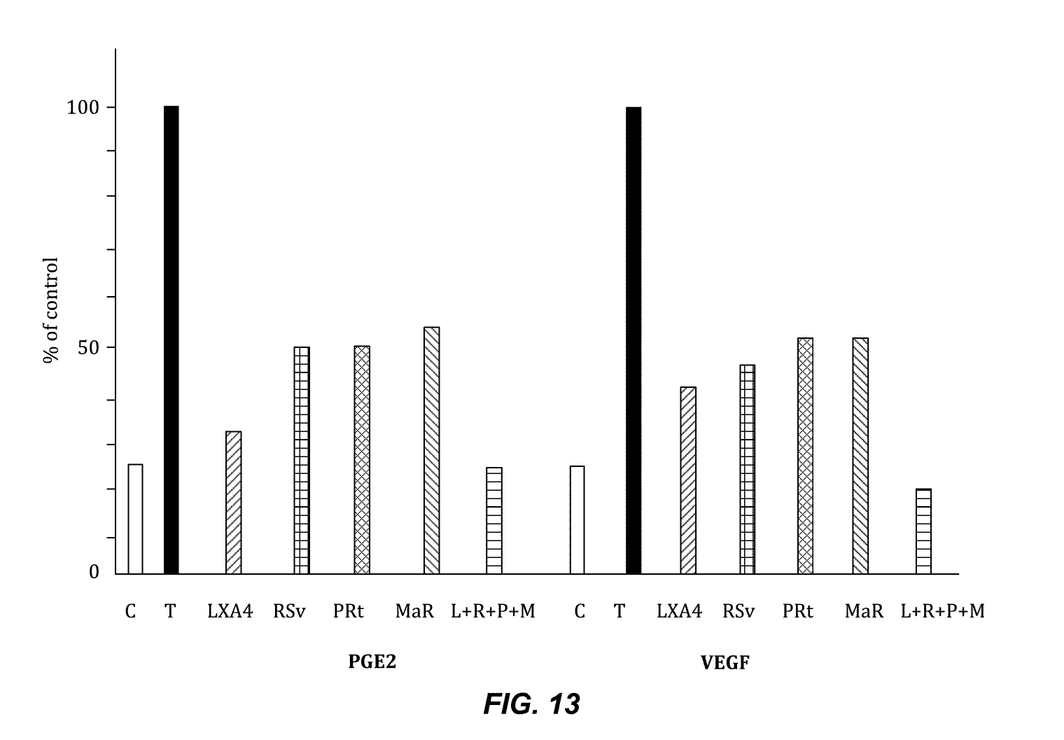

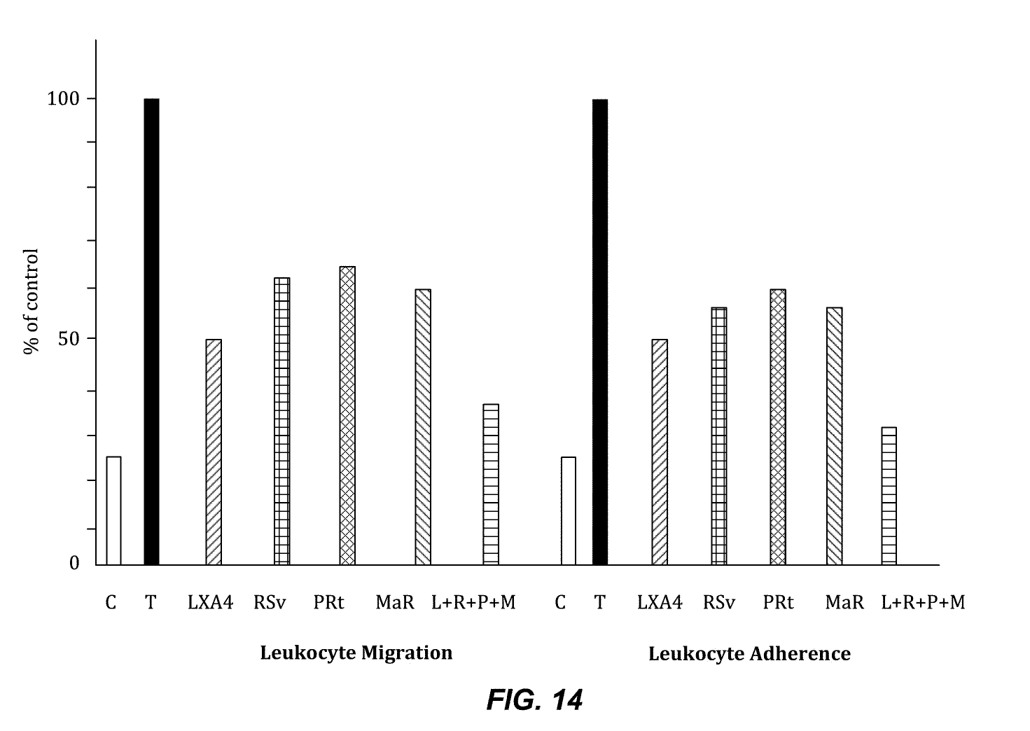

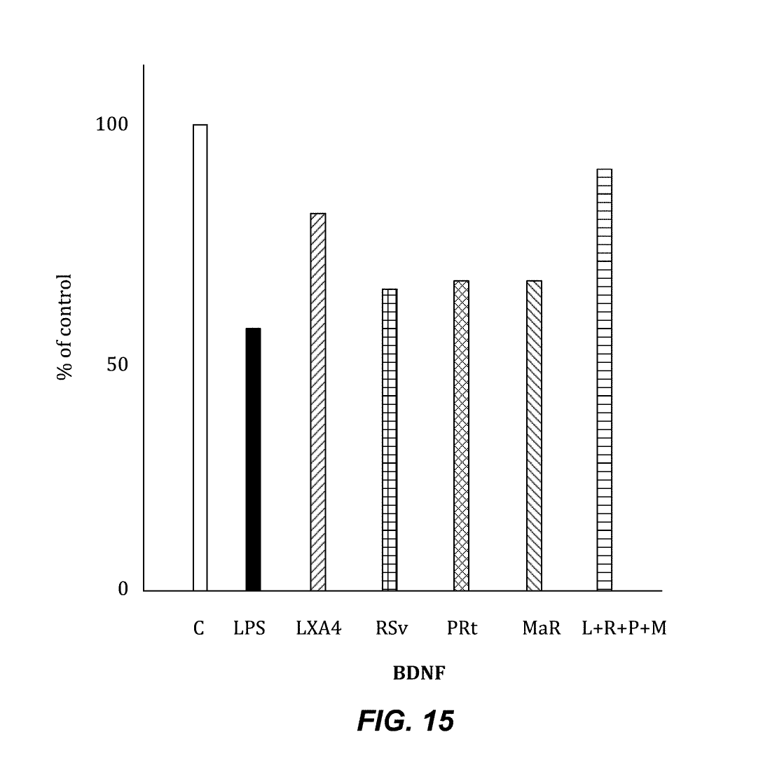

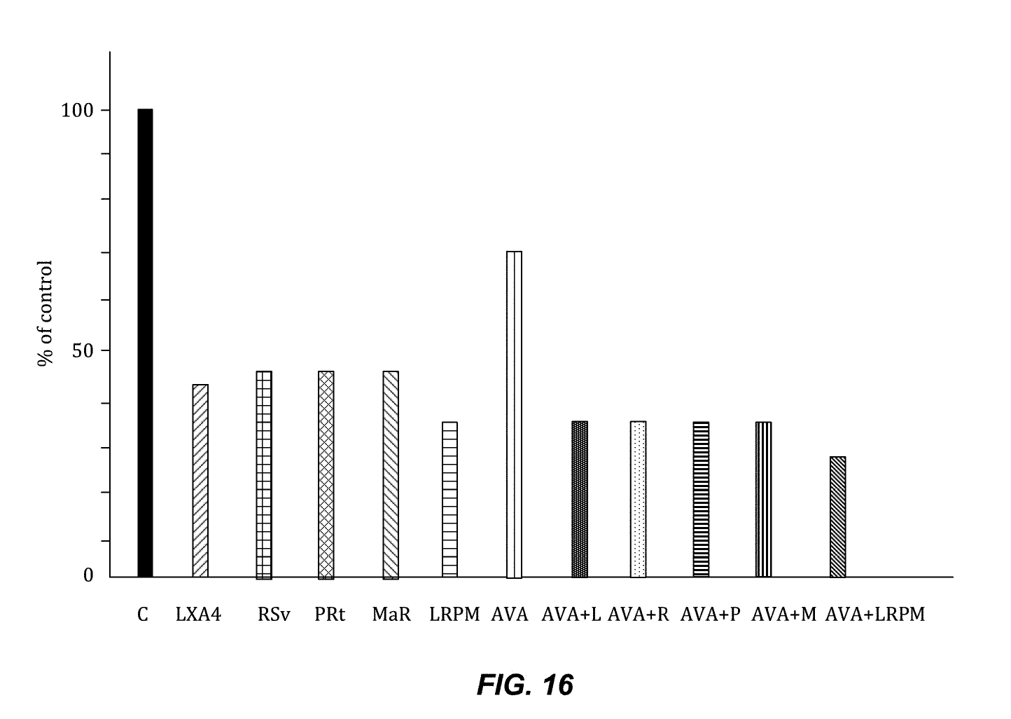

In another study, this was similar to the study given in Examples 5 and 6, except that in this instance different bioactive lipids singly and in combination were tested in combination with or without anti-VEGF antibody. Thus, in this study, experimental animals received LXA4, resolvin E1, resolvin D1, protectin D1, maresin 1 or a combination of LXA4+resolvin E1+protectin D1+maresin 1 (in the ratio of 1:1:1:1)±anti-VEGF antibody were given with appropriate control groups. In this study, dose of bioactive lipids used was 5 ng, 10 ng, 15 ng, 20 ng, 50 ng, 100 ng, 200 ng, 500 ng, 1 μg, 5 μg, or 25.0 μg once in 4 weeks for 3 months. In this study also and at all the doses tested and for different periods of time a substantial regression of DR, AMD, DME, retinopathy of prematurity was noted and the best results were obtained with bioactive lipids. A summary of the results described in examples 5, 6, and 7 are given in FIG. 16. It is evident from these results that all bioactive lipids are capable of regressing/inhibiting/arresting DR, AMD, DME and retinopathy of prematurity. A summary of the results obtained with bioactive lipids on angiogenesis (FIG. 10) in vitro; on STZ-induced cytotoxicity to vascular endothelial cells (FIG. 11); on LPS-induced IL-6 and TNF-α secretion by human macrophages in vitro (FIG. 12); on PGE2 and VEGF secretion by LPS stimulated ARPE cells in vitro (FIG. 13); leukocyte migration and adherence to endothelial cells in vitro (FIG. 14); and BDNF secretion by ARPE cells stimulated by LPS (FIG. 15) are given in respective figures.

### Example 8

### Effect of Ethanol and Stabilizing Agent on Stability and Activity of Bioactive Lipids

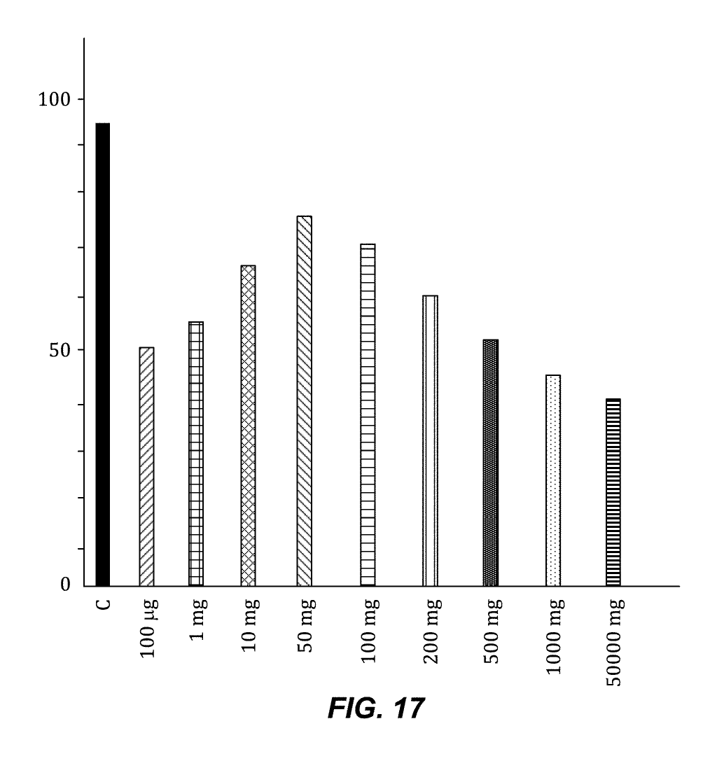

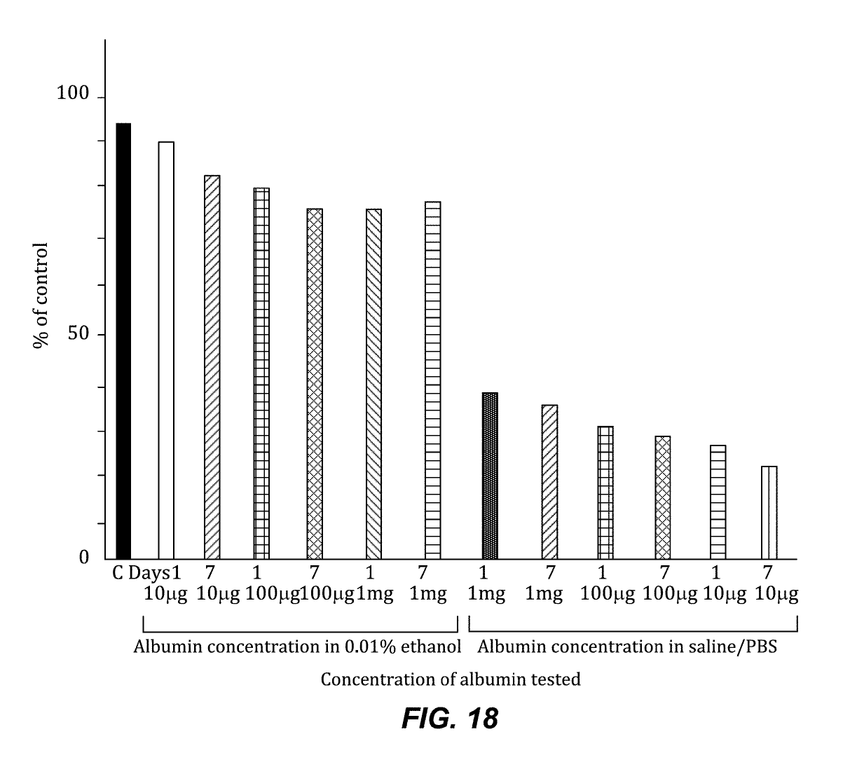

The effect of albumin as an exemplary stabilizing agent in compositions of bioactive lipids was tested as follows: To 250 ng of LXA4 dissolved in a solution of saline/PBS with or without 0.01% ethanol was added different concentrations of albumin and after a specific period of incubation the concentration of LXA4 present in the solution was tested. As shown in FIG. 18, when the concentration of albumin is between 0.01 to 0.0001% compared to LXA4 concentration, stability of LXA4 is optimum. Similarly, when the ratio between LXA4 and albumin is between 0.01% to 0.0001% LXA4 is more stable in the presence of 0.01% ethanol as shown in FIG. 17.

In addition, it was also noted that in patients with DR both plasma and vitreal levels of LXA4 and BDNF were found to be low compared to control; LXA4 enhanced the production of other bioactive lipids such as resolvins, protectins and maresins and vice versa. Thus, one bioactive lipid enhances the production and action of other bioactive lipids. In a surprise observation, inventor noted that LXA4 and other bioactive lipids function as autocrine factors enhancing their own production.

### Embodiments

Embodiment 1. A composition comprising:

one or more bioactive lipid, salt or derivative thereof, the bioactive lipid selected from the group consisting of a lipoxin, a resolvin, a protectin, and a maresin;

a stabilizing agent;

a solution selected from the group consisting of saline and phosphate buffered saline; and

ethanol;

wherein:

the concentration of the stabilizing agent and ethanol does not interfere with the anti-inflammatory, immunomodulatory and anti-angiogenic activity of the bioactive lipid.

Embodiment 2. The composition of Embodiment 1, wherein the concentration of ethanol ranges from 0.0001% to 0.01% v/v.

Embodiment 3. The composition of any one of Embodiments 1-2, wherein the bioactive lipid is selected from the group consisting of lipoxin A4, lipoxin B4, resolvin D1, resolvin E1, protectin D1, and maresin 1.

Embodiment 4. The composition of any one of Embodiments 1-3, wherein the composition comprises a lipoxin, a resolvin, a protectin, and a maresin in a ratio of 1:1:1:1, respectively.

Embodiment 5. The composition of any one of Embodiments 1-4, wherein the composition comprises lipoxin A4, resolvin E1, protectin D1, and maresin 1 in a ratio of 1:1:1:1, respectively.

Embodiment 6. The composition of any one of Embodiments 1-3, wherein the bioactive lipid is lipoxin A4.

Embodiment 7. The composition of any one of Embodiments 1-6, wherein the bioactive lipid comprises a bioactive lipid in the free acid form.

Embodiment 8. The composition of any one of Embodiments 1-7, wherein the bioactive lipid comprises a bioactive lipid salt.

Embodiment 9. The composition of Embodiment 8, wherein the bioactive lipid salt comprises a sodium salt, a magnesium salt, a manganese salt, an iron salt, a copper salt, an iodide salt, or combinations thereof.

Embodiment 10. The composition of any one of Embodiments 1-9, wherein the bioactive lipid comprises a bioactive lipid derivative.

Embodiment 11. The composition of Embodiment 10, wherein the bioactive lipid derivative comprises a glyceride, an ester, an ether, an amide, a phospholipid, an alkylated lipid, an alkoxylated lipid, a halogenated lipid, a sulfonated lipid, a phosphorylated lipid, or combinations thereof

Embodiment 12. The composition of any one of Embodiments 1-11, wherein the composition is a solution or emulsion.

Embodiment 13. The composition of any one of Embodiments 1-12, wherein the stabilizing agent is human albumin.

Embodiment 14. The composition of any one of Embodiments 1-13, wherein the concentration of the stabilizing agent ranges from 1 pg/gram to about 10 μg/gram of bioactive lipid.

Embodiment 15. The composition of any one of Embodiments 1-14, wherein the composition further comprises an anti-VEGF antibody, a corticosteroid, or combinations thereof.

Embodiment 16. The composition of Embodiment 15, wherein the anti-VEGF antibody, the corticosteroid, or both are covalently conjugated to the bioactive lipid.

Embodiment 17. The composition of any one of Embodiments 15-16, wherein the bioactive lipid, anti-VEGF antibody, and corticosteroid are present in a molar or volumetric ratio of at least 1:1:1, about 10:1:1, about 1:10:1 or about 1:1:10.

Embodiment 18. The composition of any one of Embodiments 15-17, wherein the corticosteroid is triamcinolone.

Embodiment 19. The composition of any one of Embodiments 15-16, wherein the bioactive lipid and anti-VEGF antibody are present in a molar ratio of about 2:1, about 1:1, about 1:1.5, about 1:2, or about 1:3.

Embodiment 20. The composition of any of Embodiments 1-19, wherein the concentration of bioactive lipid is at least 5%, at least 15%, at least 25%, or about 25-75% by weight.

Embodiment 21. A method for treating, preventing, or reversing diabetic retinopathy, age-related macular degeneration, retinopathy of prematurity in children, or diabetic macular edema in a mammal in need thereof, the method comprising administering to the mammal a therapeutically effective amount of a composition according to any one of Embodiments 1-20.

Embodiment 22. The method of Embodiment 21, wherein the administration comprises an intra-vitreal injection.

Embodiment 23. The method of Embodiment 21, wherein the administration comprises intra-vitreal delivery by a biodegradable wafer or membrane.

Embodiment 24. The method of any one of Embodiments 21-23, wherein the amount of bioactive lipid administered ranges from 1 ng to 100 mg in a volume ranging from 10 μL to 1000 μL.

Embodiment 25. The method of any one of Embodiments 21-24, wherein the administering comprises a single injection repeated at an interval ranging from 1 day to 6 weeks and continued for a period ranging from 4 weeks to 5 years.

Embodiment 26. The method of any one of Embodiments 21-25, wherein the method further comprises identifying and monitoring remission of diabetic retinopathy, age-related macular degeneration, retinopathy of prematurity in children, or diabetic macular edema using at least one of the following:

i) fluorescent angiogram;

ii) direct or indirect optical fundal examination of the eye;

iii) optical coherence tomography;

iv) central retinal thickness measurement; or

v) best-corrected visual acuity measurement.

Embodiment 27. The method of any one of Embodiments 21-26, wherein the administering results in at least one of the following:

i) selectively reducing the growth and inducing the apoptosis of endothelial cells that form abnormal tube-like structures, which are precursors of pathological angiogenic vessels;

ii) inhibiting the production of angiogenic factors including VEGF;

iii) blocking PGE2 production;

iv) preventing angiogenesis, including inhibiting the growth of new blood vessels;

v) suppressing inflammation locally;

vi) enhancing expression of p53;

vii) altering the expression of Bcl-2 and BAX;

viii) increasing production of lipoxin A4; or

ix) reducing abnormal angiogenesis.

Embodiment 28. A method for preparing a composition comprising:

dissolving one or more bioactive lipid, salt or derivative thereof, the bioactive lipid selected from the group consisting of a lipoxin, a resolvin, a protectin, and a maresin in ethanol to make a first mixture;

diluting the first mixture in a solution comprising saline or phosphate buffered saline thereby forming a second mixture, the second mixture having a concentration of ethanol ranging from 0.0001% to 0.01%; and adding a stabilizing agent.

1. A composition comprising:
one or more bioactive lipid, salt or derivative thereof, the bioactive lipid selected from the group consisting of a lipoxin, a resolvin, a protectin, and a maresin;
a stabilizing agent;
a solution selected from the group consisting of saline and phosphate buffered saline; and
ethanol;
wherein:
the concentration of the stabilizing agent and ethanol does not interfere with the anti-inflammatory, immunomodulatory and anti-angiogenic activity of the bioactive lipid.

2. The composition of claim 1, wherein the concentration of ethanol ranges from 0.0001% to 0.01% v/v.

3. The composition of claim 1, wherein the bioactive lipid is selected from the group consisting of lipoxin A4, lipoxin B4, resolvin D1, resolvin E1, protectin D1, and maresin 1.

4. The composition of claim 1, wherein the composition comprises a lipoxin, a resolvin, a protectin, and a maresin in a ratio of 1:1:1:1, respectively.

5. The composition of claim 1, wherein the composition comprises lipoxin A4, resolvin E1, protectin D1, and maresin 1 in a ratio of 1:1:1:1, respectively.

6. The composition of claim 1, wherein the bioactive lipid is lipoxin A4.

7. The composition of claim 1, wherein the bioactive lipid comprises a bioactive lipid in the free acid form.

8. The composition of claim 1, wherein the bioactive lipid comprises a bioactive lipid salt.

9. The composition of claim 8, wherein the bioactive lipid salt comprises a sodium salt, a magnesium salt, a manganese salt, an iron salt, a copper salt, an iodide salt, or combinations thereof

10. The composition of claim 1, wherein the bioactive lipid comprises a bioactive lipid derivative.

11. The composition of claim 10, wherein the bioactive lipid derivative comprises a glyceride, an ester, an ether, an amide, a phospholipid, an alkylated lipid, an alkoxylated lipid, a halogenated lipid, a sulfonated lipid, a phosphorylated lipid, or combinations thereof.

12. The composition of claim 1, wherein the composition is a solution or emulsion.

13. The composition of claim 1, wherein the stabilizing agent is human albumin.

14. The composition of claim 1, wherein the concentration of the stabilizing agent ranges from 1 pg/gram to about 10 μg/gram of bioactive lipid.

15. The composition of claim 1, wherein the composition further comprises an anti-VEGF antibody, a corticosteroid, or combinations thereof

16. The composition of claim 15, wherein the anti-VEGF antibody, the corticosteroid, or both are covalently conjugated to the bioactive lipid.

17. The composition of claim 15, wherein the bioactive lipid, anti-VEGF antibody, and corticosteroid are present in a molar or volumetric ratio of at least 1:1:1, about 10:1:1, about 1:10:1 or about 1:1:10.

18. The composition of claim 15, wherein the corticosteroid is triamcinolone.

19. The composition of claim 15, wherein the bioactive lipid and anti-VEGF antibody are present in a molar ratio of about 2:1, about 1:1, about 1:1.5, about 1:2, or about 1:3.

20. The composition of claims 1, wherein the concentration of bioactive lipid is at least 5%, at least 15%, at least 25%, or about 25-75% by weight.

21. A method for treating, preventing, or reversing diabetic retinopathy, age-related macular degeneration, retinopathy of prematurity in children, or diabetic macular edema in a mammal in need thereof, the method comprising administering to the mammal a therapeutically effective amount of a composition according to claim 1.

22. The method of claim 21, wherein the administration comprises an intra-vitreal injection.

23. The method of claim 21, wherein the administration comprises intra-vitreal delivery by a biodegradable wafer or membrane.

24. The method of claim 21, wherein the amount of bioactive lipid administered ranges from 1 ng to 100 mg in a volume ranging from 10 μL to 1000 μL.

25. The method of claim 21, wherein the administering comprises a single injection repeated at an interval ranging from 1 day to 6 weeks and continued for a period ranging from 4 weeks to 5 years.

26. The method of claim 21, wherein the method further comprises identifying and monitoring remission of diabetic retinopathy, age-related macular degeneration, retinopathy of prematurity in children, or diabetic macular edema using at least one of the following:
i) fluorescent angiogram;
ii) direct or indirect optical fundal examination of the eye;
iii) optical coherence tomography;
iv) central retinal thickness measurement; or
v) best-corrected visual acuity measurement.

27. The method of claim 21, wherein the administering results in at least one of the following:
i) selectively reducing the growth and inducing the apoptosis of endothelial cells that form abnormal tube-like structures, which are precursors of pathological angiogenic vessels;
ii) inhibiting the production of angiogenic factors including VEGF;
iii) blocking PGE2 production;
iv) preventing angiogenesis, including inhibiting the growth of new blood vessels;
v) suppressing inflammation locally;
vi) enhancing expression of p53;
vii) altering the expression of Bcl-2 and BAX;
viii) increasing production of lipoxin A4; or
ix) reducing abnormal angiogenesis.

28. A method for preparing a composition comprising:
dissolving one or more bioactive lipid, salt or derivative thereof, the bioactive lipid selected from the group consisting of a lipoxin, a resolvin, a protectin, and a maresin in ethanol to make a first mixture;
diluting the first mixture in a solution comprising saline or phosphate buffered saline thereby forming a second mixture, the second mixture having a concentration of ethanol ranging from 0.0001% to 0.01%; and
adding a stabilizing agent.
